(19) United States
(12) Patent Application Publication
(10) Pub. No.: US 2022/0323416 A1
(43) Pub. Date: Oct. 13, 2022

(54) Ferroportin-Inhibitors For The Use In The Prevention And Treatment Of Kidney Injuries

(71) Applicant: Vifor (International) AG, St. Gallen (CH)

(72) Inventors: Vania Manolova, St. Gallen (CH); Franz Dürrenberger, St. Gallen (CH)

(21) Appl. No.: 17/627,867

(22) PCT Filed: Jul. 17, 2020
(86) PCT No.: PCT/EP2020/070392

§ 371 Date: Jan. 18, 2022

(30) Foreign Application Priority Data
Jul. 19, 2019 (EP) 19187247.2
May 12, 2020 (EP) 20174154.3

(51) Int. Cl. A61K 31/4184; A61K 31/4439; A61P 13/12

(52) U.S. Cl. CPC A61K 31/4184; A61K 31/4439; A61P 13/12

(57) ABSTRACT

The invention relates to the use of ferroportin inhibitor compounds of the general formula (I) for preventing and treating kidney injuries, such as in particular acute kidney injuries, and the symptoms and pathological conditions associated therewith.

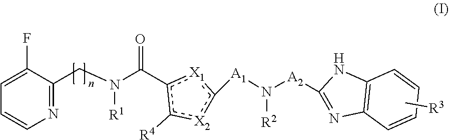

### INTRODUCTION

The invention relates to the use of compounds of the general formula (I), which act as ferroportin inhibitors, for preventing and treating kidney injuries, such as in particular acute kidney injuries, and the symptoms and pathological conditions associated therewith.

### BACKGROUND AND PRIOR ART

Iron is an essential trace element for almost all organisms and is relevant in particular with respect to growth and the formation of blood. The balance of the iron metabolism is in this case primarily regulated on the level of iron recovery from haemoglobin of ageing erythrocytes, from iron stores in the liver and the duodenal absorption of dietary iron. The released iron is taken up via the intestine, in particular via specific transport systems (DMT-1, ferroportin), transferred into the blood circulation and thereby conveyed to the appropriate tissues and organs (transferrin, transferrin receptors). In the human body, the element iron is of great importance, inter alia for oxygen transport, oxygen uptake, cell functions such as mitochondrial electron transport, cognitive functions, etc. and ultimately for the entire energy metabolism. Mammalian organisms are unable to actively discharge iron. The iron metabolism is substantially controlled by hepcidin via the cellular release of iron from macrophages, hepatocytes and enterocytes. Hepcidin acts on the absorption of iron via the intestine and via the placenta and on the release of iron from the reticuloendothelial system. The formation of hepcidin is regulated in direct correlation to the organisms iron level, i.e. if the organism is supplied with sufficient iron and oxygen, more hepcidin is formed, if iron and oxygen levels are low, or in case of increased erythropoiesis less hepcidin is formed. In the small intestinal mucosal cells and in the macrophages hepcidin binds to the transport protein ferroportin, which conventionally transports iron from the interior of the cell into the blood. The transport protein ferroportin is a transmembrane protein consisting of 571 amino acids which is expressed in the liver, spleen, kidneys, heart, intestine and placenta. In particular, ferroportin is localized in the basolateral membrane of intestinal epithelial cells. Ferroportin localized in this way thus acts to export the dietary iron into the blood. If hepcidin binds to ferroportin, ferroportin is transported into the interior of the cell, where its breakdown takes place so that the release of iron from the cells is then almost completely blocked. If the ferroportin is inactivated or inhibited, by hepcidin, so that it is unable to export the iron which is stored in the mucosal cells, the absorption of iron in the intestine is blocked. A decrease of hepcidin results in an increase of active ferroportin, thus allowing an enhanced release of stored iron and an enhanced dietary iron absorption, thus increasing the serum iron level. In pathological cases an increased iron level leads to chronic iron overload.

Besides chronic iron overload disturbed iron metabolism also causes other severe pathological conditions. The major portion of iron exists bound to hemoglobin and to proteins such as transferrin, ferritin, neutrophil gelatinase-associated lipocalin (NGAL) or in the ferric (Fe³⁺) state. Under pathological conditions highly reactive and toxic ferrous iron (Fe²⁺) can be formed. Iron fractions not bound to transferrin (or to the other traditional iron binding molecules like ferritin, haem, apoferritin, hemosiderin etc.) are collectively referred to as free iron or non-transferrin bound iron (NTBI). Further, “catalytic iron” or “labile iron” is widely known as a transitional pool of extracellular and intracellular iron, which is often loosely bound to serum albumin or endogenous chelators, such as citrate, acetate, malate, phosphate and adenine nucleosides. Labile iron exists primarily in ferrous (Fe²⁺) form. A particular detrimental aspect of such excess free iron and of catalytic or labile iron is described to lead to the undesired formation of radicals. In particular the iron (II) ions catalyze the formation (inter alia via Fenton reaction) of reactive oxygen species (ROS). These ROS cause damage to DNA, lipids, proteins and carbohydrates, including lipid peroxidation, endothelial injury, protein oxidation, mitochondrial injury and erythrocyte damage, which has far-reaching effects in cells, tissue and organs. The formation of ROS is well known and described in the literature to cause the so-called oxidative stress. NTBI and catalytic iron is widely described to exhibit high propensity to induce such ROS having the toxic potential for cellular damage, with the major organs being influenced by iron toxicity comprising heart, pancreas, kidney and organs involved in hematopoiesis. NTBI accumulation in the plasma is further considered to lead to intra-vascular damage of senescent red blood cells and thus to iron mediated intra-vascular hemolysis. Iron mediated intra-vascular hemolysis is considered to induce renal injury.

Accordingly, iron overload is known to cause tissue and organ damage, such as e.g. cardiac, liver and endocrine damage (Patel M. et al. “Non Transferrin Bound Iron: Nature, Manifestations and Analytical Approaches for Estimation” Ind. J. Clin Biochem., 2012; 27(4): 322-332 and Brissot P. et al. Review “Non-transferrin bound iron: A key role in iron overload and iron toxicity” Biochimica et Biophysica Acta, 2012; 1820, 403-410).

In particular, catalytic or labile iron has been described to be implicated in the pathogenesis of kidney injuries, e.g. via the formation of ROS and its damaging potential on kidney tissue. It has further been described, that catalytic or labile iron, as well as NTBI, act as mediators of cell death and the ensuing inflammatory response during renal ischemia-reperfusion injury (IRI) or ischemic injury, which leads to acute kidney injury (AKI). The formation of ROS by catalytic free iron causes more tissue injury leading to the release of cell-free heme and other iron-containing products and thus to a self-sustaining release of catalytic free iron, thus implicating a critical injury pathway in many acute illnesses, such as myocardial infarction, sepsis, stroke, reperfusion injury and acute kidney injury, etc.. Ischemic injury is a major cause of AKI and AKI is associated with increased morbidity, mortality and prolonged hospitalization compared to patients without such a condition. Acute ischemia leads to ATP depletion, tubular epithelial injury and hypoxic cell death. Further acute surgical situations such as operations may induce catalytic free iron via surgical stress. E.g. during operations that require cardiopulmonary bypass extracorporeally circulating blood exposed to nonphysiologic surfaces and/or shear forces may injure red blood cells, releasing free hemoglobin and free iron. Accordingly, acute surgery may lead to increased catalytic free iron, which in turn contributes to the development of AKI.′ So far, hepcidin has been described as a potential therapeutic opportunity to mitigate ischemic injury and thus AKI by modulating systemic iron homeostasis (S. Swaminathan “Iron Homeostasis Pathways as Therapeutic Targets in Acute Kidney Injury”, Nephron Clinical Practice, 2018; Scindia et al. “Iron Homeostasis in Healthy Kidney and its Role in Acute Kidney Injury”, Seminars in Nephrology, Vol. 39, No. 1, pp 76-84, 2019; Scindia et al. “Hepcidin Mitigates Renal Ischemia-Reperfusion Injury by Modulating Systemic Iron Homeostasis”, J. Am. Soc. Nephrol., 26, 2008-2814, 2015; Chawla et al. “Therapeutic Opportunities for Hepcidin in Acute Care Medicine, Grit Care Clin, 35, 357-374, 2019).

The review article of Ueda and Takasawa “Role of Hepcidin-25 in Chronic Kidney Disease: Anemia and Beyond”, Current Medicinal Chemistry, 2017, 24, 1417-1452 describes the role of Hepcidin-25 in the pathogenesis and progression of kidney injury via modulation of iron-mediated oxidant injury.

It has further been described, that NTBI and free hemoglobin accumulates in red blood cell (RBC) transfusions, in particular in stored RBC transfusions. It has been discussed that RBC transfusions may be associated with extravascular hemolysis leading to accumulation of NTBI. Based on this, RBC transfusions can be considered as a potential contributor to AKI by increasing NTBI and catalytic iron levels in transfused patients [Baek J H, et al, “Iron accelerates hemoglobin oxidation increasing mortality in vascular diseased guinea pigs following transfusion of stored blood” JClInsights, 2 (9), 2017].

WO2015/042515 describes the use of hepcidin and hepcidin derivatives for protecting kidneys from IRI.

Also iron chelation is discussed as a therapeutic approach in treating AKI (Leaf et al. “Catalytic iron and acute kidney injury”, Am J Physiol Renal Physiol. 311(5), F871-F876, 2016).

WO2018/067857 describes the use of specific compounds acting as modulators of peroxisome proliferator-activated receptor delta (PPARδ) for treating acute kidney injury by regulating mitochondria biosynthesis.

J. H. Baek et al. “Ferroportin inhibition attenuates plasma iron, oxidant stress, and renal injury following red blood cell transfusion in guinea pigs”; Transfusion, 2020 March; 60(3):513-523 report results in the attenuation of cellular injury by intravenously administering the small-molecule ferroportin inhibitor VIT-2653, provided by Vifor (International) Ltd., immediately after acute red blood cell transfusions in a model with guinea pigs.

Further, low molecular weight compounds having activity as ferroportin inhibitors and their use for treating chronic iron overload by oral administration are described in the international applications WO2017/068089 and WO2017/068090. Further, international application WO2018/192973 relates to specific salts of selected ferroportin inhibitors described in WO2017/068089 and WO2017/068090. The ferroportin inhibitors described in said three international applications overlap with the compounds according to formula (I) used in the new medical indication of the present invention. Therein, the suitability of the new ferroportin inhibitor compounds for the use in the prophylaxis and/or treatment of formation of radicals, reactive oxygen species (ROS) and oxidative stress caused by excess iron or iron overload has been described generally as well as in the prophylaxis and/or treatment of cardiac, liver and endocrine damage caused by excess iron or iron overload. However, the prophylaxis and treatment of acute ischemic situations and in particular ischemic renal injury and/or AKI are not described therein.

### OBJECT OF THE INVENTION

The object of the present invention is to provide a new method and novel drugs for preventing and treating kidney injuries, such in particular renal ischemia-reperfusion injury (herein also abbreviated as “IRI”) and acute kidney injuries, including in particular acute kidney injury (herein also abbreviated as “AKI”), renal ischemia-reperfusion injury and AKI caused by ischemic injury, AKI following surgery or surgical intervention, such as in particular following cardiac surgery most often with procedures involving cardiopulmonary bypass, other major chest or abdominal surgery, and kidney injury associated with RBC transfusion. In a further aspect an object of the invention can be seen in providing compounds for preventing and treating the kidney injuries described herein with novel drugs, which are easier and cheaper to prepare than drugs based on recombinant engineered proteins or genetically engineered drug compounds.

### DESCRIPTION OF THE INVENTION

The inventors of the present invention surprisingly found that compounds of the general formula (I) as defined herein, which act as ferroportin inhibitor (FpnI), can be used for preventing and treating the kidney injuries described herein.

Accordingly, a first aspect of the present invention relates to compounds according to formula (I) below for use in the treatment of kidney injuries, preferably kidney injuries treatment of kidney injuries induced by catalytic free iron and/or ROS:

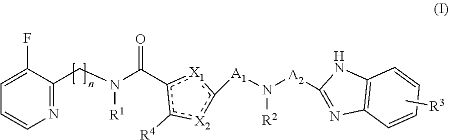

wherein

### X¹ is N or O; and

### X² is N, S or O;

with the proviso that X¹ and X² are different;
R¹ is selected from the group consisting of
hydrogen and
optionally substituted alkyl;
   n is an integer of 1 to 3;
   A¹ and A² are independently selected from the group of alkanediyl

### R² is

hydrogen, or
optionally substituted alkyl;
   or
   A¹ and R² together with the nitrogen atom to which they are bonded form an optionally substituted 4- to 6-membered ring;
   R³ indicates 1, 2 or 3 optional substituents, which may independently be selected from the group consisting of
   halogen,
cyano,
optionally substituted alkyl,
optionally substituted alkoxy, and
a carboxyl group;
   R⁴ is selected from the group consisting of
hydrogen,
halogen,
C₁-C₃-alkyl, and
halogen substituted alkyl;
   including also pharmaceutically acceptable salts, solvates, hydrates and polymorphs thereof.

### Indication

The present invention relates to the new medical use of the compounds of the formula (I) and its salts, solvates, hydrates and polymorphs, as described herein, for preventing and treating kidney injuries, which are selected from kidney injuries induced by catalytic free iron.

In a preferred aspect of the invention the kidney injuries are selected from renal ischemia-reperfusion injury (IRI), ischemic injury and acute kidney injuries.

In a further preferred aspect of the invention the kidney injuries are selected from acute kidney injury (AKI), renal ischemia-reperfusion injury (IRI), ischemic injury and AKI caused by ischemic injury, AKI following surgery or surgical intervention, such as in particular following cardiac surgery most often with procedures involving cardiopulmonary bypass, other major chest or abdominal surgery, and kidney injury associated with RBC transfusion.

The present invention thus further relates to a new method of preventing and treating the kidney injuries described herein by administering to a patient in need thereof one or more of the compounds of the formula (I) as defined herein, including its pharmaceutically acceptable salts, solvates, hydrates and polymorphs.

The new use and method of treatment according to the present invention comprises the administration of the compounds of the formula (I) as defined herein, including its pharmaceutically acceptable salts, solvates, hydrates and polymorphs to patients

The term “treat”, “treatment” or “treating” in the context of the new use of the present invention includes amelioration of at least one symptom of or pathological condition associated with the kidney injuries described herein.

The term “prophylaxis”, “prevent”, “prevention” or “preventing” in the context of the present invention includes the protection from ischemic renal injury, avoidance of occurrence of AKI or at least reducing the severity of AKI following ischemic injury, RBC transfusion or a surgery intervention e.g. by administering the compounds of the present invention prior to or accompanying or shortly after an ischemic event, RBC transfusion or the surgery intervention to prevent or at least attenuate occurrence of kidney injuries induced by catalytic free iron.

As described above, free catalytic iron or labile iron or NTBI is considered as a main cause of kidney injury, such as in particular AKI triggered by ischemia. The administration of the ferroportin inhibitor compounds of formula (I) according to the present invention helps to protect against the damaging effects of catalytic free iron. It is assumed that the ferroportin inhibitors of the present invention prevent the formation of catalytic free iron or NTBI by sequestering iron in macrophages of liver and spleen as explained in more detail below, therewith reducing its levels in plasma and reducing the risk of ROS formation. The compounds of the formula (I) of the present invention, which act as ferroportin inhibitors, therewith have the potential to prevent the noxious effects by sequestrating iron in macrophages and therefore interrupting the vicious cycle of self-sustaining release of catalytic free iron.

The inventors of the present invention found that the compounds of the formula (I) of the present invention are particularly suitable for the prevention and treatment of the kidney injuries described herein by limiting the iron availability for formation of NTBI. It has further been found that the compounds of the formula (I) of the present invention are particularly suitable for the prevention and treatment of the kidney injuries described herein by limiting reactive oxygen species (ROS) to avoid kidney tissue injury.

Further to catalytic free iron. NTBI and LPI must be considered to cause kidney injuries. NTBI encompasses all forms of serum iron that are not tightly associated with transferrin and is chemically and functionally heterogeneous. LPI (Labile Plasma Iron) represents a component of NTBI that is both redox active and chelatable, capable of permeating into organs and inducing tissue iron overload.

The following parameters can be determined to evaluate the efficacy of the compounds of the present invention in the new medical use: plasma creatinine, glomerular filtration rate (including estimated glomerular filtration rate eGFR), urine albumin excretion, urine neutrophil gelatinase-associated lipocalin (NGAL), NTBI, LPI, RBC hemolysis, blood urea nitrogen (BUN), plasma hemoglobin (Hb), total plasma iron, plasma hepcidin, renal neutrophil infiltration, serum IL-6, spleen, kidney and/or liver iron content, renal ferroportin, KIM-1 (Kidney Injury Molecule-1) as an acute marker for kidney injury in blood and urine, and H-ferritin.

Additionally or alternatively, the efficacy of the compounds of the present invention can be determined via the kidney tubular injury score, such as e.g. the CSA-NGAL score (Cardiac Surgery Associated NGAL Score) for detecting acute tubular damage as described in more detail below, the KDIGO score described in more detail below or the EGTI score comprising Endothelial, Glomerular, Tubular and Interstitial (EGTI) components to evaluate histology [described e.g. by: Khalid et al. “Kidney ischaemia reperfusion injury in the rat: the EGTI scoring system as a valid and reliable tool for histological assessment” Journal of Histology & Histopathology, Vol. 3, 2016].

The determination of the aforesaid parameters can be carried out using conventional methods of the art, in particular by those described below in more detail. The compounds (I) of the present invention are suitable to correct or improve at least one of these parameters.

In preventing or treating AKI in particular the following parameters are improved by administering the compounds of the formula (I):

The new treatment may result in a decrease of serum creatinine (sCr) in the patient by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or at least 100% and/or by an accelerated decrease and/or an increased extend of decrease of the sCr value, determined at any time point within a time period of up to one week, up to 6 days, up to 5 days, up to 4 days, up to 84 hours, up to 72 hours, up to 60 hours, up to 48 hours, up to 36 hours, up to 24 hours, or up to 12 hours following the first administration and/or following an ischemic event and as compared to the sCr levels in the patient determined at any time point within 12 hours, 24 hours, 36 hours, 48 hours, 1 week, 2 weeks, 3 weeks, or 4 weeks prior to the commencement of treatment of the invention. sCr concentration can be determined by conventional methods, such as according to an assay described in the Examples below,

In a further aspect, the new treatment may result in corrected (decreased) urine albumin excretion in the patient by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95% or at least 100%, determined at any time point within a time period of up to one week, up to 6 days, up to 5 days, up to 4 days, up to 84 hours, up to 72 hours, up to 60 hours, up to 48 hours, up to 36 hours, up to 24 hours, or up to 12 hours following the first administration and/or following an ischemic event and as compared to the urine albumin excretion in the patient determined at any time point within 12 hours, 24 hours, 36 hours, 48 hours, 1 week, 2 weeks, 3 weeks, or 4 weeks prior to the commencement of treatment of the invention. Urine albumin excretion can be determined by conventional methods.

The new treatment may result in a decrease of blood urea nitrogen (BUN) in the patient by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or at least 100%, determined at any time point within a time period of up to one week, up to 6 days, up to 5 days, up to 4 days, up to 84 hours, up to 72 hours, up to 60 hours, up to 48 hours, up to 36 hours, up to 24 hours, or up to 12 hours following the first administration and/or following an ischemic event and as compared to the BUN levels in the patient determined at any time point determined at any time point within 12 hours, 24 hours, 36 hours, 48 hours, 1 week, 2 weeks, 3 weeks, or 4 weeks prior to the commencement of treatment of the invention. BUN concentration can be determined by conventional methods, such as according to an assay described in the Examples below.

The new treatment may result in a decrease of total plasma iron in the patient by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or at least 100%, determined at any time point within a time period of up to one week, up to 6 days, up to 5 days, up to 4 days, up to 84 hours, up to 72 hours, up to 60 hours, up to 48 hours, up to 36 hours, up to 24 hours, or up to 12 hours following the first administration and/or following an ischemic event and as compared to the total plasma iron levels in the patient determined at any time point within 12 hours, 24 hours, 36 hours, 48 hours, 1 week, 2 weeks, 3 weeks, or 4 weeks prior to the commencement of treatment of the invention. Total plasma iron concentration can be determined by conventional methods, such as according to an assay described in the Examples below.

The new treatment may result in a decrease of interleukin-6 (IL-6) levels in the patient by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or at least 100%, determined at any time point within a time period of up to one week, up to 6 days, up to 5 days, up to 4 days, up to 84 hours, up to 72 hours, up to 60 hours, up to 48 hours, up to 36 hours, up to 24 hours, or up to 12 hours following the first administration and/or following an ischemic event and as compared to the total IL-6 levels in the patient determined at any time point within 12 hours, 24 hours, 36 hours, 48 hours, 1 week, 2 weeks, 3 weeks, or 4 weeks prior to the commencement of treatment of the invention. IL-6 concentration can be determined by conventional methods, such as according to an assay described in the Examples below.

The new treatment may result in a decrease of KIM-1 levels in the patient by at least 1%, 2%, 3%, 4%, 5%, 10%, 15%, 20%, 25%. 30%, 35%, 40%, 45%, 50%, 55%, 60%, 65%, 70%, 75%, 80%, 85%. 90%, 95%, or at least 100%, determined at any time point within a time period of up to 72 hours, up to 60 hours, up to 48 hours, up to 36 hours, up to 24 hours, or up to 12, 8, 6, 5, 4, 3, 2, 1 and 0.5 hours following the first administration and/or following an ischemic event and as compared to the total KIM-1 levels in the patient determined at any time point within 0.5, 1, 2, 3, 4, 5, 6, 8, 12, 24, 36, or 48 hours, or up to <1 week prior to the commencement of treatment of the invention. KIM-1 concentration can be determined by conventional methods, such as according to an assay described in the Examples below.

The new treatment may result in an increase in spleen and/or liver iron concentration, in the patient by at least 1%, 2%, 3%, 4%, 5%. 10%, 15%, 20%, 25%. 30%, 35%, 40%, 45%, 50%, 55%, 60%, 65%, 70%, 75%, 80%, 85%, 90%, 95%, or at least 100%, determined at any time point within a time period of up to one week, up to 6 days, up to 5 days, up to 4 days, up to 84 hours, up to 72 hours, up to 60 hours, up to 48 hours, up to 36 hours, up to 24 hours, or up to 12 hours following the first administration and/or following an ischemic event and as compared to the levels of spleen and liver iron concentration in the patient determined at any time point within 12 hours, 24 hours, 36 hours, 48 hours, 1 week, 2 weeks, 3 weeks, or 4 weeks prior to the commencement of treatment of the invention. Spleen and liver iron concentration can be determined by conventional methods, such as described in the Examples below.

The new treatment may result in a decrease in kidney iron concentration in the patient by at least 1%, 2%, 3%, 4%, 5%, 10%, 15%, 20%, 25%, 30%, 35%. 40%, 45%, 50%, 55%, 60%, 65%, 70%, 75%, 80%, 85%, 90%, 95%, or at least 100%, determined at any time point within a time period of up to one week, up to 6 days, up to 5 days, up to 4 days, up to 84 hours, up to 72 hours, up to 60 hours, up to 48 hours, up to 36 hours, up to 24 hours, or up to 12 hours following the first administration and/or following an ischemic event and as compared to the levels of kidney iron concentration in the patient determined at any time point within 12 hours, 24 hours, 36 hours, 48 hours, 1 week, 2 weeks, 3 weeks, or 4 weeks prior to the commencement of treatment of the invention. Kidney iron concentration can be determined by conventional methods, such as described in the Examples below.

As explained by Patel et al. (2012; cited above) in normal physiological conditions the level of transferrin is sufficient for complete scavenging of free iron, ensuring the absence of NTBI and accordingly NTBI levels in normal healthy individuals do not exceed 1 μmol/L and are mostly undetectable by most common methods. In the absence of transferrin NTBI levels up to 20 μmol/L were reported and in the presence of insufficient transferrin NTBI levels up to 10 μmol/L have been found. However, as described by Patel et al. (2012) and Brissot et al. (2012) the determination strongly depends from the applied method and assays used and the technical difficulties resulting from the determination of heterogeneous chemical forms of circulating NTBI must be taken into account. For example, fluorescent measurements with a repeatable accuracy down to 0.1 μM/L have been described by Hider et al. (2010) cited by Brissot et al. (2012). According to Patel et al. (2012; Table 1) elevated NTBI levels in clinical iron overload conditions range between 0.25 to 4.0 μmol/L (with varying accuracy and varying determination methods). Considering this, in the sense of the present invention NTBI levels are considered as elevated if detectable with the known methods (e.g. those described in Patel et al. (2012) or in Brissot et al. (2012), preferably when exceeding 0.1 μm/L.

In a particular aspect, the new treatment of the present invention results in reduced NTBI levels in a patient by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or at least 100%, determined at any time point within a time period of up to 72 hours, up to 60 hours, up to 48 hours, up to 36 hours, up to 24 hours, or up to 12, 8, 6, 5, 4, 3, 2, 1 and 0.5 hours following the first administration and/or following an ischemic event and as compared to the total NTBI levels in the patient determined at any time point within 0.5, 1, 2, 3, 4, 5, 6, 8, 12, 24, 36, or 48 hours, or up to <1 week prior to the commencement of treatment of the invention. NTBI can be determined by conventional methods, such as according to an assay described below.

In a particular aspect, the new treatment of the present invention results in reduced LPI levels in a patient by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or at least 100%, determined at any time point within a time period of up to 72 hours, up to 60 hours, up to 48 hours, up to 36 hours, up to 24 hours, or up to 12, 8, 6, 5, 4, 3, 2, 1 and 0.5 hours following the first administration and/or following an ischemic event and as compared to the total LPI levels in the patient determined at any time point within 0.5, 1, 2, 3, 4, 5, 6, 8, 12, 24, 36, or 48 hours, or up to <1 week prior to the commencement of treatment of the invention. LPI can be determined by conventional methods, such as according to an assay described in the Examples below.

The new treatment may result in an inhibition of tubular injury, such as tubular necrosis.

The new treatment may result in an inhibition of apoptosis.

The new treatment may result in a reduced IRI-induced renal neutrophil infiltration.

In a further aspect, the new treatment of the present invention results in reduced ROS levels in kidney tissue of the patients by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%, or at least 100%, determined at any time point within a time period of up to 5 days, up to 6 days, up to 7 days, up to 8 days, up to 9 days, up to 10 days, up to 11 days, up to 12 days, up to 13 days, up to 14 days, up to 15 days, up to 16 days, up to 17 days, up to 18 days, up to 19 days, up to 20 days, up to 21 days and up to 1 month following the first administration and/or following an ischemic event and as compared to the ROS levels in kidney tissue of the patient determined at any time point within 12 hours, 24 hours, 36 hours, 48 hours, 1 week, 2 weeks, 3 weeks, or 4 weeks prior to the commencement of treatment of the invention. ROS levels can be determined by conventional methods, such as according to an assay described in the Examples below, such as in particular as described by Scindia et al., 2015 (cited above).

In a further aspect, the new treatment may result in corrected (increased) kidney H-ferritin levels in the patient by at least 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95% or at least 100%, determined at any time point within a time period of up to one week, up to 6 days, up to 5 days, up to 4 days, up to 84 hours, up to 72 hours, up to 60 hours, up to 48 hours, up to 36 hours, up to 24 hours, or up to 12 hours following the first administration and/or following an ischemic event and as compared to the kidney H-ferritin levels in the patient determined at any time point within 12 hours, 24 hours, 36 hours, 48 hours, 1 week, 2 weeks, 3 weeks, or 4 weeks prior to the commencement of treatment of the invention. Kidney H-ferritin levels can be determined by conventional methods, such as according to an assay described in the Examples below.

With the new method of treatment according to the present invention, one or more of the aforesaid improvements can be achieved.

In particular, with the new method of treatment the occurrence of AKI, renal ischemia-reperfusion injury and AKI caused by ischemic injury, AKI following surgery or surgical intervention, such as in particular following cardiac surgery most often with procedures involving cardiopulmonary bypass, other major chest or abdominal surgery, and kidney injury associated with RBC transfusion can be reduced.

In a particular aspect of the new treatment according to the present invention the abnormal change of one or more of the above described parameters or indicators of ischemic injury and (acute) kidney injuries is inhibited by the administration of the compounds of the formula (I).

Thus, in a further aspect the invention relates to compounds of the formula (I), or its salts, solvates, hydrates and polymorphs, for the use of preventing or treating kidney injuries as described herein, wherein the prophylaxis and/or treatment comprises
a) decrease, accelerated decrease or prevention of increase of serum creatinine and/or
b) increase or prevention of decrease of estimated glomerular filtration rate (eGFR) and/or
c) decrease or prevention of increase of renal ferroportin and/or
d) increase or prevention of decrease of H-ferritin levels and/or
e) decrease or prevention of increase of renal neutrophil infiltration and/or
f) decrease or prevention of increase of serum IL-6 levels.

### Patient Group

The subjects to be treated in the new use according to the invention can be any mammals such as rodents and primates, and in a preferred aspect the new medical use relates to the treatment of humans. The subjects to be treated with the new method according to the invention are also designated as “patients”.

The subjects to be treated can be of any age. One aspect of the invention relates to the treatment of children and adolescents. Accordingly, in a preferred aspect of the invention the subjects to be treated with the new methods described herein are less than 18 years old. More particularly, the subjects to be treated with the new methods described herein are less than 16 years old, less than 15 years old, less than 14 years old, less than 13 years old, less than 12 years old, less than 11 years old, less than 10 years old, less than 9 years old, less than 8 years old, less than 7 years old, less than 6 years old, or less than 5 years old. In a further aspect of the invention the subjects to be treated with the new methods described herein are 1-3 years old, 3-5 years old, 5-7 years old, 7-9 years old, 9-11 years old, 11-13 years old, 13-15 years old, 15-20 years old, 20-25 years old, 25-30 years old, or greater than 30 years old. In the case of treating adults, the subjects to be treated with the new methods described herein are 18-25 years old, 20-25 years old, 25-30 years old, 30-35 years old, 35-40 years old, 40-45 years old, 45-50 years old, 50-55 years old, 55-60 years old, or greater than 60 years old. In the case of treating elderly patients the subjects to be treated with the new methods described herein are 60-65 years old, 65-70 years old, 70-75 years old, 75-80 years old, or greater than 80 years old.

In a further aspect of the invention the subjects to be treated are characterized by having increased plasma creatinine levels and/or a decreased estimated glomerular filtration rate (eGFR) compared to normal physiological levels. Normal range of blood creatinine is 0.84 to 1.21 mg/dL (74.3 to 107 μM/L).

Further, one or more of the following parameters can be used to characterize subjects to be treated:
a) Urine albumin excretion and/or
b) Neutrophil gelatinase-associated lipocalin (NGAL) and/or
c) detectable NTBI levels and/or
d) RBC hemolysis levels, and/or
e) blood urea nitrogen (BUN) levels, and/or
f) plasma hemoglobin (Hb) levels, and/or
g) total plasma iron levels, and/or
h) plasma hepcidin levels, and/or
i) renal neutrophil infiltration levels and/or
j) serum IL-6 levels and/or
k) spleen, kidney and/or liver iron levels
   In subjects to be treated according to the invention one or more of said parameters deviates from normal physiological levels as determined with conventional diagnostic methods.

Said parameters can be used to determine a patient group suffering from AKI or being at risk of developing AKI.

In a further aspect of the invention the patient group or population suffering from ischemic injury or AKI or being at risk of developing AKI and to be treated with the new method according to the invention are selected from subjects (patients) having elevated NTBI levels. NTBI levels are considered as elevated, if detectable with the known methods as discussed above. Preferably, NTBI levels ≥0.1 μM/L are considered as elevated in patients. Possible determination methods are described e.g. in de Swart et al. “Second international round robin for the quantification of serum non-transferrin-bound iron and labile plasma iron in patients with iron-overload disorders” Haematologica, 2016; 101(1): 38-45.

Similarly, LPI levels are considered as elevated, if detectable with the known methods as discussed above and the determination methods as described in de Swart et al. “Second international round robin for the quantification of serum non-transferrin-bound iron and labile plasma iron in patients with iron-overload disorders” Haematologica, 2016; 101(1): 38-45 can be used for determination.

Usually, the serum creatinine level (sCr) is used to classify the severity and form of AKI.

According to the classification of KDIGO (2012) the following specific criteria for the diagnosis of AKI have been established, wherein AKI can be diagnosed if any one of the following is present:

Increase in SCr by ≥0.3 mg/dl (≥26.5 μmol/l) within 48 hours; or

Increase in SCr to ≥1.5 times baseline, which has occurred within the prior 7 days; or

Urine volume <0.5 ml/kg/h for 6 hours

Further a classification system according to the RIFLE/AKIN criteria, proposed by the Acute Dialysis Quality Initiative (ADQI) group, aids in assessment of the severity of a person's acute kidney injury. The acronym RIFLE is used to define the spectrum of progressive kidney injury seen in AKI:

| RIFLE | AKIN | | |
|---|---|---|---|
| Stage | Stage | Serum creatinine/GFR | urine output |
| Risk | 1 | 1.5-fold increase in the serum | <0.5 mL/kg |
| | | creatinine, or glomerular filtration | per hour |
| | | rate (GFR) decrease by 25 percent | for six hours |
| Injury | 2 | Two-fold increase in the serum | <0.5 mL/kg |
| | | creatinine, or GFR decrease by 50 | per hour |
| | | percent | for 12 hours |
| Failure | 3 | Three-fold increase in the serum | <0.3 mL/kg |
| | | creatinine, or GFR decrease by 75 | per hour for |
| | | percent | 24 hours, or |
| | | | no urine |
| | | | output (anuria) |
| | | | for 12 hours |
| Loss | | Complete loss of kidney function | |
| | | (e.g., need for renal replacement | |
| | | therapy) for more than four weeks | |
| End-stage | | Complete loss of kidney function | |
| kidney | | (e.g., need for renal replacement | |
| disease | | therapy) for more than three months | |

A further important marker for acute kidney injury is the estimated Glomerular Filtration Rate (eGFR), which is a test of measuring the level of kidney function. The eGFR is calculated from the blood creatinine values, considering age, body size and ender of the patient. Decreased GFR compared to normal levels indicate that kidneys are not working as well as they should. In adults, the normal eGFR is >90. eGFR declines with age, even in people without kidney disease. The average estimated eGFR based on age can be considered as follows:

| Age (years) | Average estimated eGFR |
|---|---|
| 20-29 | 116 |
| 30-39 | 107 |
| 40-49 | 99 |
| 50-59 | 93 |
| 60-69 | 85 |
| 70+ | 75 |

Further, acute tubular damage can be used as an early diagnostic marker of AKI by using the CSA-NGAL score. This score is based on NGAL as the biomarker for defining acute tubular damage, originally developed in connection with cardiac surgery-associated acute kidney injury (CSA-AKI), however the score can be adopted to determine tubular damage in AKI in general:

| Concentration | | or Delta (Δ) NGAL | |
|---|---|---|---|
| Sample [ng/mL] | | at following measurement | CSA-NGAL Score |
| uNGAL | <50 | | 0: tubular damage |
| pNGAL | <100 | | unlikely |
| uNGAL | 50-<150 | | 1: tubular damage |
| pNGAL | 100-<200 | | possible |
| uNGAL | 150-<1000 | Δ >100 + second value ≥125 | 2: tubular damage |
| pNGAL | 200-<1000 | Δ >100 + second value ≥150 | |
| uNGAL | >1000 | | 3: severe tubular |
| pNGAL | >1000 | | damage |

As a further possibility, but rarely used in clinical practice, biopsy can be performed, in particular if there is a need for diagnosis of the underlying cause. Then, the above mentioned EGTI score based on endothelial, glomerular, tubular and interstitial components can be used to evaluate histology according to Table 1 of Khalid et al., 2016 (cited above):

TABLE 1
The EGTI histology scoring system.

| Tissue type | Damage | Score |
|---|---|---|
| Tubular | No damags | 0 |
| | Loss of Brush Border (BB) in less than 25% of | 1 |
| | tubular cells. Integrity of basal membrane. | |
| | Loss of BB in me than 25% of tubular cells, | 2 |
| | Thickened basal membrane | |
| | (Plus) Inflammation, Cast formation, | 3 |
| | Necrosis up to 60% of tubular cells | |
| | (Plus) Necrosis in more than 60% of tubular | 4 |
| | cells | |
| Endothelal | No Damage | 0 |
| | Endothelial swelling | 1 |
| | Endothelial disruption | 2 |
| | Endothelial loss | 3 |
| Glomerular | No damage | 0 |
| | Thickening of Bowman capsule | 1 |
| | Retraction of glomerular tu [symbol] | 2 |
| | Glomerular fibrosis | 3 |
| Tubulo/ | No damage | 0 |
| Interstitial | Inflammation [symbol] haemorrhage in less than 25% | 1 |
| | of tissue | |
| | (Plus) necrosis in less than 25% of tissue | 2 |
| | Necrosis up to 60% | 3 |
| | Necrosis more than 60% | 4 |

[symbol]

indicates data missing or illegible when filed

Considering this, in a further aspect of the invention the patient group or population to be treated with the new method of the present invention is suffering from AKI or being at risk of suffering from AKI in any of the stages defined by the KDIGO or RIFLE/AKIN classification or by reduced eGFR levels or having a CSA-NGAL score >0, or having an EGTI histology score >0.

In particular aspect of the invention the patients to be treated are characterized by
i) having increased plasma creatinine levels and/or
ii) increased urine albumin excretion and/or
iii) a decreased estimated glomerular filtration rate (eGFR), each compared to normal physiological levels, and/or
iv) the patients are classified to suffer from AKI or to be at risk of suffering from AKI by any of the stages defined by the KDIGO or RIFLE/AKIN classification or by a CSA-NGAL score >0, or by an EGTI histology score >0.

### Administration Forms

In a further aspect of the invention the prevention or treatment of kidney diseases as defined herein, such as in particular IRI or ischemic injury and AKI, may comprise the oral and/or the intravenous administration of one or more of the compounds of the formula (I), its salts, solvates, hydrates or polymorphs, each as described anywhere herein, to a patient in need thereof.

Oral administration may preferably be selected in cases of a prophylactic treatment, e.g. prior to a planned surgical intervention. Intravenous administration may preferably be selected in the case of acute occurrence of ischemic events or in hospital.

For oral administration, the compounds of the formula (I) according to the invention are preferably provided in medicaments or pharmaceutical compositions in the form of oral administration forms, including e.g. pills, tablets, such as enteric-coated tablets, film tablets and layer tablets, sustained release formulations for oral administration, depot formulations, dragees, granulates, emulsions, dispersions, microcapsules, microformulations, nanoformulations, liposomal formulations, capsules, such as enteric-coated capsules, powders, microcrystalline formulations, epipastics, drops, ampoules, solutions and suspensions for oral administration.

In a preferred embodiment thereof, the compounds of the formula (I) according to the invention are administered in the form of a tablet or capsule, as defined above. These may be present, for example, as acid resistant forms or with pH dependent coatings.

Accordingly, a further aspect of the present invention relates to the compounds of the formula (I) according to the invention, including pharmaceutically acceptable salts, solvates, hydrates and polymorphs thereof, as well as medicaments, compositions and combined preparations comprising the same for the use in the prophylaxis and treatment of kidney injuries as defined herein in the form of oral administration forms.

Parenteral administration includes e.g. subcutaneous or intravenous administration, with intravenous administration being preferred, and accordingly the compounds of the formula (I) according to the invention are preferably provided in medicaments or pharmaceutical compositions in the form of injectable administration forms, including e.g. ampoules, solutions, suspensions, infusion solutions or injection solutions etc.

Accordingly, a further aspect of the present invention relates to the compounds of the formula (I) according to the invention, including pharmaceutically acceptable salts, solvates, hydrates and polymorphs thereof, as well as medicaments, compositions and combined preparations comprising the same for the use in the prophylaxis and treatment of kidney injuries as defined herein in the form of injectable, preferable intravenous, administration forms.

### Dosing Regimen

A further aspect of the invention relates to the compounds of the formula (I) according to the invention for the use according to the present invention, wherein the treatment is characterized by one of the following dosing regimens:

In one aspect the compounds of the formula (I) according to the invention can be administered to a patient in need thereof in a dose of 0.001 to 500 mg, for example 1 to 4 times a day. However, the dose can be increased or reduced depending on the age, weight, condition of the patient, severity of the disease or type of administration. In a further aspect of the invention the compounds of the formula (I) can be administered as a dose of 0.1 mg, 0.2 mg, 0.3 mg, 0.4 mg, 0.5 mg, 0.6 mg, 0.7 mg, 0.8 mg, 0.9 mg, 1 mg, 1.5 mg, 2 mg, 2.5 mg, 3 mg, 3.5 mg, 4 mg, 4.5 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 25 mg, 30 mg, 35 mg, 40 mg, 45 mg, 50 mg, 55 mg, 60 mg, 65 mg, 70 mg, 75 mg, 80 mg, 85 mg, 90 mg, 95 mg, 100 mg, 105 mg, 110 mg, 115 mg, 120 mg, 125 mg, 130 mg, 135 mg, 140 mg, 145 mg, 150 mg, 155 mg, 160 mg, 165 mg, 170 mg, 175 mg, 180 mg, 185 mg, 190 mg, 195 mg, 200 mg, 205 mg, 210 mg, 215 mg, 220 mg, 225 mg, 230 mg, 235 mg, 240 mg, 245 mg, 250 mg, 255 mg, 260 mg, 265 mg, 270 mg, 275 mg, 280 mg, 285 mg, 290 mg, 295 mg, 300 mg, 325 mg, 350 mg, 375 mg, 400 mg, 425 mg, 450 mg, 475 mg, 500 mg.

Preferred is a dose of between 0.5 to 500 mg, more preferred between 1 to 300 mg, more preferred between 1 to 250 mg. Most preferred is a dose of 5 mg, 15 mg, 60 mg, 120 mg or 240 mg.

Further preferred is a dose between 0.001 to 35 mg/kg body weight, between 0.01 to 35 mg/kg body weight, between 0.1 to 25 mg/kg body weight, or between 0.5, 1, 2, 3, 4, 5, 6, 7, 8, 9, 10 and up to 20 mg/kg body weight.

It is possible to administer the above defined dosages as a total daily dose either in a single dose daily or divided into sub-doses for administration twice or more times daily.

Even more preferred is a dose of 120 mg for patients with >50 kg body weight and of 60 mg for patients with <50 kg body weight, in each case once or twice daily. In a further aspect it is possible to select one of the above defined dosages as an initial dose and subsequently administer 1 or more times the same or varying doses of those defined above in repeating intervals of 1 to 7 days. 1 to 5 days, preferably of 1 to 3 days, or every second day.

The initial dose and the subsequent doses can be selected among the above defined dosages and adjusted/varied in accordance with the need of the patient within the provided ranges.

In particular, the amount of subsequent doses can be appropriately selected depending on the individual patient, the course of disease and the treatment response. It is possible to administer 1, 2, 3, 4, 5, 6, 7, and more subsequent doses.

It is possible that the initial dose is equal or different to the one or more subsequent doses. It is further possible, that the subsequent doses are equal or different.

The repeating intervals can be of the same length or can be varied depending on the individual patient, the course of disease and the treatment response.

Preferably, the subsequent doses are of decreasing amount with increasing number of subsequent dosing.

In the case of oral administration a suitable dose of between 3 mg and 300 mg, more preferred between 5 mg and 250 mg, most preferred of 5 mg, 15 mg. 60 mg, 120 mg or 240 mg is administered once daily over a treatment period of at least 3 days, at least 5 days, at least 7 days. In a further preferred aspect a dose of 60 mg or 120 mg is administered orally once daily. In a further preferred aspect a total daily dose of 120 mg is administered orally by administering twice daily a 60 mg dose.

In a further preferred aspect a total daily dose of 240 mg is administered orally by administering twice daily a 120 mg dose. Said doses turned out to be safe and well tolerated.

In the case of intravenous administration a suitable dose of between 5 to 300 mg, e.g. of 5 to 50 mg, 5 to 40 mg, 5 to 30 mg, 5 to 20 mg, 5 to 10 mg, or of 50 to 300 mg, 50 to 250 mg, 50 to 200 mg, 50 to 150 mg, 50 to 100 mg, or 100 to 300 mg is administered. Said intravenous doses can be administered e.g. once, twice or more times daily and a treatment period of at least 1 day, at least 2 days, at least 3 days, at least 5 days, at least 7 days can be chosen depending on the severity the patients overall condition and the treatment success.

In a further aspect the compounds of the formula (I) for the use in the new method described herein the prophylaxis and/or treatment comprises the administration of one or more of the compounds of the formula (I), its salts, solvates, hydrates or polymorphs, to a patient in need thereof one or more times within a time period of >0 to 48 hours, >0 to 36 hours, >0 to 24 hours, >0 to 20 hours, >0 to 18 hours, >0 to 16 hours, >0 to 12 hours, >0 to 10 hours, >0 to 8 hours, >0 to 6 hours, >0 to 5 hours, >0 to 4 hours, >0 to 3 hours, >0 to 2 hours, >0 to 1 hour, >0 to 0.5 hours prior to IRI or ischemic injury, prior to RBC transfusion, prior to surgery or a surgical intervention, such as e.g. cardiac surgery, including procedures involving cardiopulmonary bypass, other major chest or abdominal surgery.

In a further aspect the compounds of the formula (I) for the use in the new method described herein the prophylaxis and/or treatment comprises the administration of one or more of the compounds of the formula (I), its salts, solvates, hydrates or polymorphs, to a patient in need thereof one or more times within a time period between immediately after and up to 48 hours after an ischemic reperfusion event, RBC transfusion or a surgical intervention, preferably between immediately after up to 12 hours after an ischemic reperfusion event, RBC transfusion or a surgical intervention.

In oral dosing fast oral absorption with detectable levels as early as 15 to 30 minutes post-dose have been observed. The absorption level can be maintained stable even upon repeated dosing and no critical accumulation is observed.

The preferred dosing regimen further turned out to efficiently decrease mean serum iron levels and mean calculated transferrin saturation and to shift the mean serum hepcidin peak, indicating its efficiency for treating AKI.

In a further aspect of the invention, the initial and one or more subsequent dosing is adjusted depending on the sCr concentration of the treated patient. The sCr concentration is determined with conventional methods.

### Ferroportin (Fpn) Inhibitor Compounds

The present invention relates to the new medical use of the compounds of the formula (I) as defined herein:

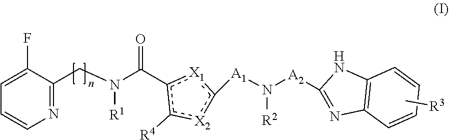

Therein and throughout the invention, the substituent groups have the meaning as defined in detail anywhere herein:

Optionally substituted alkyl preferably includes: linear or branched alkyl preferably containing 1 to 8, more preferably 1 to 6, particularly preferably 1 to 4, even more preferred 1, 2 or 3 carbon atoms, also being indicated as C₁-C₄-alkyl or C₁-C₄-alkyl.

Optionally substituted alkyl further includes cycloalkyl containing preferably 3 to 8, more preferably 5 or 6 carbon atoms.

Examples of alkyl residues containing 1 to 8 carbon atoms include: a methyl group, an ethyl group, an n-propyl group, an i-propyl group, an n-butyl group, an 1-butyl group, a sec-butyl group, a t-butyl group, an n-pentyl group, an i-pentyl group, a sec-pentyl group, a t-pentyl group, a 2-methylbutyl group, an n-hexyl group, a 1-methylpentyl group, a 2-methylpentyl group, a 3-methylpentyl group, a 4-methylpentyl group, a 1-ethylbutyl group, a 2-ethylbutyl group, a 3-ethylbutyl group, a 1,1-dimethylbutyl group, a 2,2-dimethylbutyl group, a 3,3-dimethylbutyl group, a 1-ethyl-1-methylpropyl group, an n-heptyl group, a 1-methylhexyl group, a 2-methylhexyl group, a 3-methylhexyl group, a 4-methylhexyl group, a 5-methylhexyl group, a 1-ethylpentyl group, a 2-ethylpentyl group, a 3-ethylpentyl group, a 4-ethylpentyl group, a 1,1-dimethylpentyl group, a 2,2-dimethylpentyl group, a 3,3-dimethylpentyl group, a 4,4-dimethylpentyl group, a 1-propylbutyl group, an n-octyl group, a 1-methylheptyl group, a 2-methylheptyl group, a 3-methylheptyl group, a 4-methylheptyl group, a 5-methylheptyl group, a 6-methylheptyl group, a 1-ethylhexyl group, a 2-ethylhexyl group, a 3-ethylhexyl group, a 4-ethylhexyl group, a 5-ethylhexyl group, a 1,1-dimethylhexyl group, a 2,2-dimethylhexyl group, a 3,3-dimethylhexyl group, a 4,4-dimethylhexyl group, a 5,5-dimethylhexyl group, a 1-propylpentyl group, a 2-propylpentyl group, etc. Those containing 1 to 4 carbon atoms (C₁-C₄-alkyl), such as in particular methyl, ethyl, n-propyl, i-propyl, n-butyl, 1-butyl, sec-butyl, and t-butyl are preferred. C₁-C₃ alkyl, in particular, methyl, ethyl, propyl and i-propyl are more preferred. Most preferred are C₁ and C₂ alkyl, such as methyl and ethyl.

Cycloalkyl residues containing 3 to 8 carbon atoms preferably include: a cyclopropyl group, a cyclobutyl group, a cyclopentyl group, a cyclohexyl group, a cycloheptyl group and a cyclooctyl group. A cyclopropyl group, a cyclobutyl group, a cyclopentyl group and a cyclohexyl group are preferred. A cyclopropyl group is particularly preferred.

Substituents of the above-defined optionally substituted alkyl preferably include 1, 2 or 3 of the same or different substituents, selected, for example, from the group consisting of: halogen as defined below, such as preferably F, cycloalkyl as defined above, such as preferably cyclopropyl, optionally substituted heteroaryl as defined below, such as preferably a benzimidazolyl group, optionally substituted amino as defined below, such as preferably an amino group or benzyloxycarbonylamino, a carboxyl group, an aminocarbonyl group as defined below, as well as an alkylene group such as in particular a methylene-group, forming for example a methylene-substituted ethyl-group (CH₃—(C═CH₂)— or

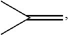

wherein * indicates the binding site).

Within the meaning of the present invention, halogen includes fluorine, chlorine, bromine and iodine, preferably fluorine or chlorine, most preferred is fluorine.

Examples of a linear or branched alkyl residue substituted by halogen and containing 1 to 8 carbon atoms include:

a fluoromethyl group, a difluoromethyl group, a trifluoromethyl group, a chloromethyl group, a dichloromethyl group, a trichloromethyl group, a bromomethyl group, a dibromomethyl group, a tribromomethyl group, a 1-fluoroethyl group, a 1-chloroethyl group, a 1-bromoethyl group, a 2-fluoroethyl group, a 2-chloroethyl group, a 2-bromoethyl group, a difluoroethyl group such as a 1,2-difluoroethyl group, a 1,2-dichloroethyl group, a 1,2-dibromoethyl group, a 2,2-difluoroethyl group, a 2,2-dichloroethyl group, a 2,2-dibromoethyl group a 2,2,2-trifluoroethyl group, a heptafluoroethyl group, a 1-fluoropropyl group, a 1-chloropropyl group, a 1-bromopropyl group, a 2-fluoropropyl group, a 2-chloropropyl group, a 2-bromopropyl group, a 3-fluoropropyl group, a 3-chloropropyl group, a 3-bromopropyl group, a 1,2-difluoropropyl group, a 1,2-dichloropropyl group, a 1,2-dibromopropyl group, a 2,3-difluoropropyl group, a 2,3-dichloropropyl group, a 2,3-dibromopropyl group, a 3,3,3-trifluoropropyl group, a 2,2,3,3,3-pentafluoropropyl group, a 2-fluorobutyl group, a 2-chlorobutyl group, a 2-bromobutyl group, a 4-fluorobutyl group, a 4-chlorobutyl group, a 4-bromobutyl group, a 4,4,4-trifluorobutyl group, a 2,2,3,3,4,4,4-heptafluorobutyl group, a perfluorobutyl group, a 2-fluoropentyl group, a 2-chloropentyl group, a 2-bromopentyl group, a 5-fluoropentyl group, a 5-chloropentyl group, a 5-bromopentyl group, a perfluoropentyl group, a 2-fluorohexyl group, a 2-chlorohexyl group, a 2-bromohexyl group, a 6-fluorohexyl group, a 6-chlorohexyl group, a 6-bromohexyl group, a perfluorohexyl group, a 2-fluoroheptyl group, a 2-chloroheptyl group, a 2-bromoheptyl group, a 7-fluoroheptyl group, a 7-chloroheptyl group, a 7-bromoheptyl group, a perfluoroheptyl group, etc. Fluoroalkyl, difluoroalkyl and trifluoroalkyl are mentioned in particular, and trifluoromethyl and mono- and di-fluoroethyl is preferred. Particularly preferred is trifluoromethyl.

Examples of a cycloalkyl-substituted alkyl group include the above-mentioned alkyl residues containing 1 to 3, preferably 1 cycloalkyl group such as, for example: cyclopropylmethyl, cyclobutylmethyl, cyclopentylmethyl cyclohexylmethyl, 2-cyclopropylethyl, 2-cyclobutylethyl, 2-cyclopentylethyl 2-cyclohexylethyl, 2- or 3-cyclopropylpropyl, 2- or 3-cyclobutylpropyl, 2- or 3-cyclopentylpropyl, 2- or 3-cyclohexylpropyl, etc. Preferred is cyclopropylmethyl.

Examples of a heteroaryl-substituted alkyl group include the above-mentioned alkyl residues containing 1 to 3, preferably 1 (optionally substituted) heteroaryl group, such as, for example a pyridinyl, a pyridazinyl, a pyrimidinyl, a pyrazinyl, a pyrazolyl, an imidazolyl, a benzimidazolyl, a thiophenyl, or an oxazolyl group, such as pyridine-2-yl-methyl, pyridine-3-yl-methyl, pyridine-4-yl-methyl, 2-pyridine-2-yl-ethyl, 2-pyridine-1-yl-ethyl, 2-pyridine-3-yl-ethyl, pyridazine-3-yl-methyl, pyrimidine-2-yl-methyl, pyrimidine-4-yl-methyl, pyrazine-2-yl-methyl, pyrazol-3-yl-methyl, pyrazol-4-yl-methyl, pyrazol-5-yl-methyl, imidazole-2-yl-methyl, imidazole-5-yl-methyl, benzimidazol-2-yl-methyl, thiophen-2-yl-methyl, thiophen-3-yl-methyl, 1,3-oxazole-2-yl-methyl.

Preferred is an alkyl group which is substituted with a benzimidazolyl group, such as benzimidazol-2-yl-methyl and benzimidazol-2-yl-ethyl.

Examples of an amino-substituted alkyl residue include the above-mentioned alkyl residues containing 1 to 3, preferably 1 (optionally substituted) amino group, as defined below, such as, for example, aminoalkyl (NH₂-alkyl) or mono- or dialkylamino-alkyl, such as aminomethyl, 2-aminoethyl, 2- or 3-aminopropyl, methylaminomethyl, methylaminoethyl, methylaminopropyl, 2-ethylaminomethyl, 3-ethylaminomethyl, 2-ethylaminoethyl, 3-ethylaminoethyl, etc. with 3-aminopropyl being preferred, or an alkyl group, which may be substituted with an optionally substituted alkyloxycarbonylamino group such as a group according to formula

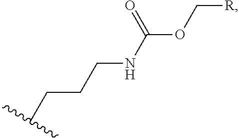

wherein R defines a phenyl group, forming a benzyloxycarbonylaminopropyl group.

Optionally substituted amino according to the invention preferably includes: amino (—NH₂), optionally substituted mono- or dialkylamino (alkyl-NH—, (alkyl)₂N—), wherein with respect to “alkyl” reference can be made to the definition of optionally substituted alkyl above. Preferred is mono- or dimethylamino, mono- or diethylamino and monopropylamino. Most preferred is an amino group (—NH₂), and monopropylamino.

Further, in the sense of the present invention, a carboxyl group indicates a group [—(C═O)—OH] and an aminocarbonyl group indicates a group [NH₂—(C═O)—].

Optionally substituted alkoxy includes an optionally substituted alkyl-O-group, wherein reference may be made to the foregoing definition of the alkyl group. Preferred alkoxy groups are linear or branched alkoxy groups containing up to 6 carbon atoms such as a methoxy group, an ethoxy group, an n-propyloxy group, an i-propyloxy group, an n-butyloxy group, an i-butyloxy group, a sec-butyloxy group, a t-butyloxy group, an n-pentyloxy group, an i-pentyloxy group, a sec-pentyloxy group, a t-pentyloxy group, a 2-methylbutoxy group, an n-hexyloxy group, an i-hexyloxy group, a t-hexyloxy group, a sec-hexyloxy group, a 2-methylpentyloxy group, a 3-methylpentyloxy group, a 1-ethylbutyloxy group, a 2-ethylbutyloxy group, a 1,1-dimethylbutyloxy group, a 2,2-dimethylbutyloxy group, a 3,3-dimethylbutyloxy group, a 1-ethyl-1-methylpropyloxy group, as well as cycloalkyloxy groups such as a cyclopentyloxy group or a cyclohexyloxy group. A methoxy group, an ethoxy group, an n-propyloxy group and an i-propyloxy group are preferred. A methoxy and ethoxy group is more preferred. Particularly preferred is a methoxy group.

Throughout the invention, optionally substituted alkanediyl is preferably a divalent straight-chained or branched alkanediyl radical having from 1 to 6, preferably from 1 to 4, more preferably 1, 2 or 3 carbon atoms, which can optionally carry from 1 to 3, preferably 1 or 2 substituents selected from the group consisting of halogen, hydroxyl (—OH), an oxo group (C═O; forming a carbonyl or acyl group [—(C═O)—]) and an alkyl group as defined above such as preferably methyl. The following may be mentioned as preferred examples: methylene, ethane-1,2-diyl, ethane-1,1-diyl, propane-1,3-diyl, propane-1,1-diyl, propane-1,2-diyl, propane-2,2-diyl, butane-1,4-diyl, butane-1,2-diyl, butane-1,3-diyl, butane-2,3-diyl, butane-1,1-diyl, butane-2,2-diyl, butane-3,3-diyl, pentane-1,5-diyl, etc. Particularly preferred is methylene, ethane-1,2-diyl, ethane-1,1-diyl, propane-1.3-diyl, propane-2,2-diyl, and butane-2,2-diyl. Most preferred are methylene, ethane-1,2-diyl and propane-1,3-diyl.

A preferred substituted alkanediyl radical is a hydroxy-substituted alkanediyl such as a hydroxy-substituted ethanediyl, an oxo-substituted alkanediyl such as an oxo-substituted methylene or ethanediyl radical, forming a carbonyl or an acyl (acetyl) group, a halogen substituted alkanediyl group such as an alkanediyl group being substituted with one or two halogen atoms selected from F and Cl, preferably 2,2-di-fluoro-ethanediyl, or an alkanediyl group which is substituted with a methyl group.

According to the present invention it is further possible that A¹, having the meaning of a linear or branched alkanediyl group as defined above, and R², having the meaning of an optionally substituted alkyl group as defined above, together with the nitrogen atom to which they are bonded form an optionally substituted 4- to 6-membered ring, which may be substituted with 1 to 3 substituents as defined above. Accordingly, A¹ and R² may together from a group according to one the following formulae

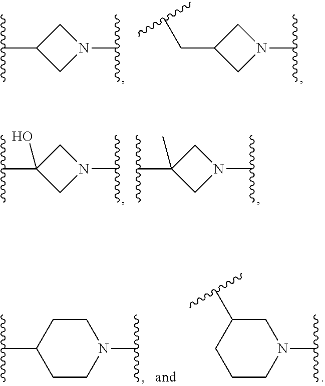

Therein a (substituted or unsubstituted) 4-membered ring-formation is preferred, such as very particularly a group

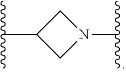 [SMILES: *C1CN(*)C1]

Therein the left-hand binding site indicates the direct binding site to the heterocyclic 5-membered ring between the positions X¹ and X² in formula (I) of the present invention. The right-hand binding site indicates the binding site to the group A² having the meaning of an alkanediyl group as defined herein.

In the formula (I) as defined anywhere herein n has the meaning of an integer of 1 to 3, including 1, 2 or 3 thus indicating a methylene-group, an ethane-1,2-diyl group or a propane-1,3-diyl group. More preferably n is 1 or 2 and even more preferably n is 1, indicating a methylene group.

In the present invention the individual substituents of the formula (I) above may have the following meaning:

### A) X¹ is N or O; and

X² is N, S or O;
with the proviso that X¹ and X² are different;
thus forming 5-membered heterocycles according to the formulae

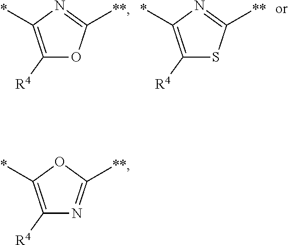

wherein * indicates the binding site to the aminocarbonyl-group and ** indicates the binding site to the A¹-group.
   B) n is an integer of 1, 2 or 3; preferably n is 1 or 2, more preferably n is 1.
   C) R¹ is selected from the group consisting of
hydrogen and
optionally substituted alkyl (as defined above);
   preferably R¹ is hydrogen or methyl, more preferably R¹ is hydrogen.
      D) R² is selected from the group consisting of
hydrogen, and
optionally substituted alkyl (as defined above);
   preferably R² is hydrogen or C₁-C₄-alkyl, more preferably R² is hydrogen or methyl, even more preferably R² is hydrogen.
      E) R³ indicates 1, 2 or 3 optional substituents, which may independently be selected from the group consisting of
halogen (as defined above),
cyano,
optionally substituted alkyl (as defined above),
optionally substituted alkoxy (as defined above), and
a carboxyl group (as defined above);
   preferably R³ indicates 1 or 2 optional substituents, which may independently be selected from the group consisting of
halogen,
cyano,
alkyl (as defined above), which may be substituted with 1, 2 or 3 halogen atoms (as defined above),
optionally substituted alkoxy (as defined above), and
a carboxyl group (as defined above);
   more preferably R³ indicates 1 or 2 optional substituents, which may independently be selected from the group consisting of
F and Cl,
cyano,
trifluoromethyl,
methoxy, and
a carboxyl group;
   even more preferably hydrogen, indicating an unsubstituted terminal benzimidazolyl-ring in formula (I).
      F) R⁴ is selected from the group consisting of
hydrogen,
halogen (as defined above),
C₁-C₃-alkyl, and
halogen substituted alkyl (as defined above);
   preferably R⁴ is selected from the group consisting of
hydrogen
Cl,
methyl, ethyl, iso-propyl, and
trifluoromethyl;
   more preferably R⁴ is selected from the group consisting of
hydrogen,
Cl,
methyl, and
trifluoromethyl;
   more preferably R⁴ is selected from the group consisting of
hydrogen,
Cl, and
methyl;
   even more preferably R⁴ is hydrogen.
      G) A¹ is alkanediyl;
   preferably A¹ is methylene or ethane-1,2-diyl, more preferably A¹ is ethane-1,2-diyl.
      H) A² is alkanediyl;
   preferably A² is methylene, ethane-1,2-diyl or propane-1,3-diyl;
   more preferably A² is methylene or ethane-1,2-diyl;
   even more preferably A² is ethane-1,2-diyl.
      I) or A¹ and R² together with the nitrogen atom to which they are bonded form an optionally substituted 4- to 6-membered ring as defined above;

therein A¹ and R² together with the nitrogen atom to which they are bonded preferably form an optionally substituted 4-membered ring as defined above;

therein A¹ and R² together with the nitrogen atom to which they are bonded more preferably form an unsubstituted 4-membered ring (azetidinyl-ring).

The substituents of the compounds of the following (I) may in particular have the following meaning:

n has any of the meanings according to B) above and the remaining substituents may have any of the meanings as defined in A) and C) to I).

R¹ has any of the meanings according to C) above and the remaining substituents may have any of the meanings as defined in A) and B) and D) to I).

R² has any of the meanings according to D) above and the remaining substituents may have any of the meanings as defined in A) to C) and E) to H) or I).

R³ has any of the meanings according to E) above and the remaining substituents may have any of the meanings as defined in A) to D) and F) to I).

R⁴ has any of the meanings according to F) above and the remaining substituents may have any of the meanings as defined in A) to E) and G) to I).

A¹ has any of the meanings according to G) above and the remaining substituents may have any of the meanings as defined in A) to F) and H) or I).

A² has any of the meanings according to H) above and the remaining substituents may have any of the meanings as defined in A) to G) and I).

R² and A¹ have any of the meanings as defined in I) and the remaining substituents may have any of the meanings as defined in A) to C), E), F) and H).

In a preferred embodiment of the present invention the compounds of general formula (I) are defined by

X′ is N or O; and

X² is N, S or 0;

with the proviso that X¹ and X² are different;

R¹ is hydrogen;

n is 1, 2 or 3;

A¹ is methylene or ethane-1,2-diyl;

A² is methylene, ethane-1,2-diyl or propane-1,3-diyl;

R² is hydrogen or C₁-C₄-alkyl;

or

A¹ and R² together with the nitrogen atom to which they are bonded form an optionally substituted 4-membered ring;

R³ indicates 1 or 2 optional substituents, which may independently be selected from the group consisting of
halogen,
cyano,
alkyl, which may be substituted with 1, 2 or 3 halogen atoms,
optionally substituted alkoxy, and
a carboxyl group;

R⁴ is selected from the group consisting of
hydrogen
Cl,
methyl, ethyl, iso-propyl, and
trifluoromethyl.

In a further preferred embodiment of the present invention the compounds of general formula (I) are defined by
X′ is or O; and
X² is N, S or O;
with the proviso that X¹ and X² are different;
R¹ is hydrogen;
n is 1 or 2;
A¹ is methylene or ethane-1,2-diyl;
A² is methylene, ethane-1,2-diyl or propane-1,3-diyl;
R² is hydrogen or methyl;
or
A¹ and R² together with the nitrogen atom to which they are bonded form an unsubstituted 4-membered ring;
R³ indicates 1 or 2 optional substituents, which may independently be selected from the group consisting of
   F and Cl,
   cyano,
   trifluoromethyl,
   methoxy, and
   a carboxyl group;
   R⁴ is selected from the group consisting of
   hydrogen,
   Cl,
   methyl, and
   trifluoromethyl.

In a further preferred embodiment of the present invention the compounds of general formula (I) are defined by
X′ is N or O; and
X² is N, S or 0;
with the proviso that X¹ and X² are different;
R¹ is hydrogen;
n is 1;
A¹ is methylene or ethane-1,2-diyl;
A² is methylene, ethane-1,2-diyl or propane-1,3-diyl;
R² is hydrogen;
or
A¹ and R² together with the nitrogen atom to which they are bonded form an unsubstituted 4-membered ring;
R³ indicates hydrogen, thus forming an unsubstituted terminal benzimidazolyl-ring;
R⁴ is selected from the group consisting of
   hydrogen,
   Cl, and
   methyl.

In a further preferred embodiment of the present invention the compounds of general formula (I) are defined by
X′ is N or O; and
X² is N, S or 0;
with the proviso that X¹ and X² are different;
R¹ is hydrogen;
n is 1;
A¹ is methylene or ethane-1,2-diyl;
A² is methylene, ethane-1,2-diyl or propane-1,3-diyl;
R² is hydrogen;
or
A¹ and R² together with the nitrogen atom to which they are bonded form an unsubstituted 4-membered ring;
R³ indicates hydrogen, thus forming an unsubstituted terminal benzimidazolyl-ring; and
R⁴ is hydrogen.

In a further aspect the present invention relates to the new use and method of treatment as defined herein, wherein the compounds according to formula (I), or its salts, solvates, hydrates and polymorphs, are selected from compounds of the formula (I) as shown above, wherein
n=1;
R³=hydrogen;
R⁴=hydrogen;
A¹=methylene or ethane-1,2-diyl;
A²=methylene, ethane-1,2-diyl or propane-1.3-diyl;
or A′ and R² together with the nitrogen atom to which they are bonded form an optionally substituted 4-membered ring,
forming compounds according to formula (II) or (III):

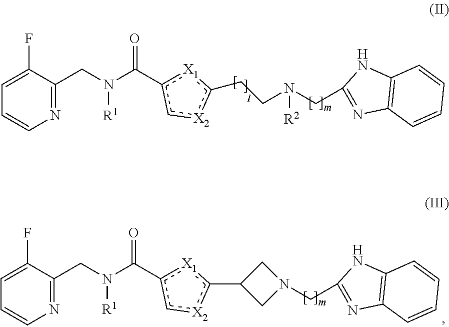

wherein in formula (II) and/or (III)

### I is 0 or 1;

m is an integer of 1, 2 or 3 and
X¹, X², R¹ and R² have the meaning as defined for compounds of formula (I) anywhere herein.

Preferably, in the formulae (II) and (III) X¹ and X² have the meaning as defined above in A).

In formula (II) R¹ and R² are preferably hydrogen.
In formula (III) R¹ is preferably hydrogen and m is preferably 2.

In a further preferred embodiment of the present invention the compounds of general formula (II) are defined by
X¹ and X² are selected from and O and are different;
R¹=hydrogen;
R²=hydrogen;
I=1; and
m=2.
   Further compounds acting as ferroportin inhibitors and being suitable in the treatment of severe forms of β-thalassemia as defined herein are those as described in WO2020/123850 A1, incorporated herein by reference in its entirety. Particular compounds among those described in WO2020/123850 A1 being suitable in the treatment of severe forms of β-thalassemia as defined herein can be selected from the group consisting of:

| | | Mass |
|---|---|---|
| | | Found |
| Structure | IUPAC Name | (M + 1) |
| 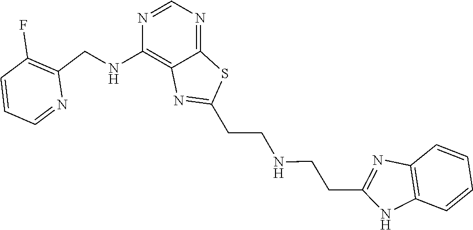 [SMILES: Fc1cccnc1CNc1ncnc2sc(CCNCCc3nc4ccccc4[nH]3)nc12] | 2-(2-{[2-(1H-1,3-benzodiazol-2- yl)ethyl]amino}ethyl)-N-[(3- fluoropyridin-2-yl)methyl]- [1,3]thiazolo[5,4-d]pyrimidin-7- amine | 449.2 |
| 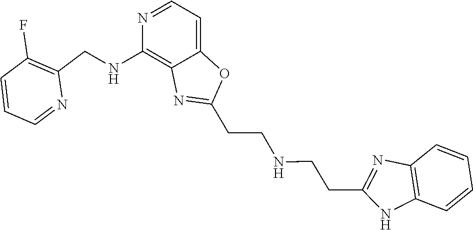 [SMILES: Fc1cccnc1CNc1nccc2oc(CCNCCc3nc4ccccc4[nH]3)nc12] | 2-(2-{[2-(1H-1,3-benzodiazol-2- yl)ethyl]amino}ethyl)-N-[(3- fluoropyridin-2-yl)methyl]- [1,3]oxazolo[4,5-c]pyridin-4- amine | 432.2 |
| 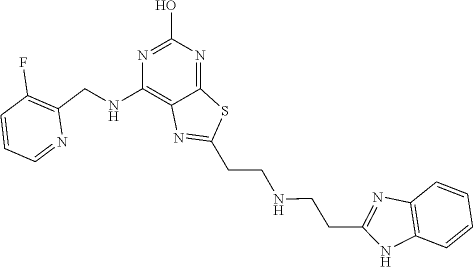 [SMILES: Oc1nc(NCc2ncccc2F)c2nc(CCNCCc3nc4ccccc4[nH]3)sc2n1] | 2-(2-{[2-(1H-1,3-benzodiazol-2- yl)ethyl]amino}ethyl)-7-{[(3- fluoropyridin-2-yl)methyl]amino}- [1,3]thiazolo[5,4-d]pyrimidin-5- ol | 465.1 |
| 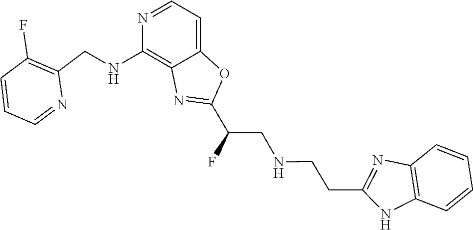 [SMILES: Fc1cccnc1CNc1nccc2oc([C@H](F)CNCCc3nc4ccccc4[nH]3)nc12] | 2-[(1R)-2-{[2-(1H-1,3- benzodiazol-2-yl)ethyl]amino}- 1-fluoroethyl]-N-[(3- fluoropyridin-2-yl)methyl]- [1,3]oxazolo[4,5-c]pyridin-4- amine | 450.2 |
| 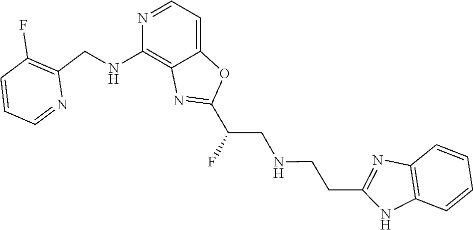 [SMILES: Fc1cccnc1CNc1nccc2oc([C@@H](F)CNCCc3nc4ccccc4[nH]3)nc12] | 2-[(1S)-2-{[2-(1H-1,3- benzodiazol-2-yl]ethyl]amino)-1- fluoroethyl]-N-[(3-fluoropyridin- 2-yl)methyl]-[1,3]oxazolo[4,5- c]pyridin-4-amine | 450.2 |
| 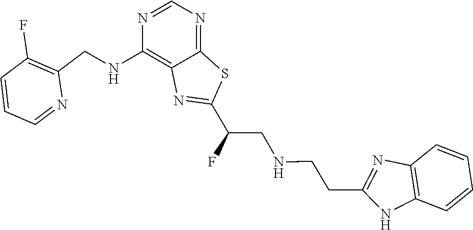 [SMILES: Fc1cccnc1CNc1ncnc2sc([C@H](F)CNCCc3nc4ccccc4[nH]3)nc12] | 2-[(1R)-2-([2-(1H-1,3- benzodiazol-2-yl)ethyl]amino}- 1-fluoroethyl]-N-[(3- fluoropyridin-2-yl)methyl]- [1,3]thiazolo[5,4-d]pyrimidin-7- amine | 467 |

In a further preferred aspect the present invention relates to the new use and method of treatment as defined herein, wherein the compounds according to formula (I) or of the compounds according to WO2020/123850 A1 are used in the form of its pharmaceutically acceptable salts, or solvates, hydrates and polymorphs thereof.

With respect to suitable pharmaceutically acceptable salts of the compounds of the formulae (I), (II) and (III) as defined anywhere herein reference is made to the international applications WO2017/068089, WO2017/068090 and in particular WO2018/192973. The definition of pharmaceutically acceptable salts as disclosed therein is herein enclosed by reference.

In a further preferred aspect the present invention relates to the new use and method of treatment as defined herein, wherein the pharmaceutically acceptable salts of the compounds of the formulae (I), (II) or (III) are selected from salts with acids from the group consisting of benzoic acid, citric acid, fumaric acid, hydrochloric acid, lactic acid, malic acid, maleic acid, methanesulfonic acid, phosphoric acid, succinic acid, sulfuric acid, tartaric acid and toluenesulfonic acid. Preferably acids from the group consisting of citric acid, hydrochloric acid, maleic acid, phosphoric acid and sulfuric acid are selected.

In a further preferred aspect the present invention relates to the new use and method of treatment as defined herein, wherein the pharmaceutically acceptable salts of the compounds of the formulae (I), (II) or (III) are selected from mono-salts (1:1 salts), triple salts (1:3 salts) and salts being characterized by a ratio of compound (I), (II) or (III) to acid of 1-2: 1-3; including solvates, hydrates and polymorphs thereof.

Therein, the salts of the compounds (I), (II) or (III) may be characterized by a selected ratio of base:acid, i.e. compound (I), (II) or (III): the acids as defined above, in the range of 1.0 to 2.0 (mol base): 1.0 to 3.0 (mol acid). In a particular embodiment the selected ratio of base:acid is 1.0 to 2.0 (mol base): 1.0 to 2.0 (mol acid).

Particular examples comprise the following ratios of base:acid, i.e. compound (I), (II) or (III): the acids as defined above:
1.0 (mol base): 1.0 (mol acid);
1.0 (mol base): 1.25 (mol acid):
1.0 (mol base): 1.35 (mol acid);
1.0 (mol base): 1.5 (mol acid);
1.0 (mol base): 1.75 (mol acid);
1.0 (mol base): 2.0 (mol acid);
1.0 (mol base): 3.0 (mol acid); and
2.0 (mol base): 1.0 (mol acid).

Therein, a salt having a ratio of base:acid of 1:1 is also called “mono-salt(s)” or “1:1 salt(s)”. For example, a mono-HCl salt is also designated as 1HCl or 1HCl salt.

Therein, a salt having a ratio of base:acid of 1:2 is also called “di-salt(s)” or “1:2 salt(s)”. For example, a di-HCl salt is also designated as 2HCl or 2HCl salt.

Therein, a salt having a ratio of base:acid of 1:3 is also called “tri-salt(s)”, “triple salts(s)” or “1:3 salt(s)”. For example, a tri-HCl salt is also designated as 3HCl or 3HCl salt.

A salt having a ratio of base:acid of 1:1.25 is also called “1:1.25 salt(s)”.

A salt having a ratio of base:acid of 1:1.35 is also called “1:1.35 salt(s)”.

A salt having a ratio of base:acid of 1:1.5 is also called “1:1.5 salt(s)”.

A salt having a ratio of base:acid of 1:1.75 is also called “1:1.75 salt(s)”.

A salt having a ratio of base:acid of 2:1 is also called “hemi-salt(s)” or “2:1 salt(s)”.

The salts of the compounds of formulae (I), (II) or (III) according to the present invention may be present in amorphous, polymorphous, crystalline and/or semi-crystalline (partly crystalline) form as well as in the form of a solvate of the salt. Preferably salts of the compounds of formulae (I), (II) or (III) according to the present invention are present in crystalline and/or semi-crystalline (partly crystalline) form and/or in the form of solvates thereof.

The preferable crystallinity of the salts or salt solvates can be determined by using conventional analytical methods, such as especially by using the various X-ray methods, which permit a clear and simple analysis of the salt compounds. In particular, the grade of crystallinity can be determined or confirmed by using Powder X-ray diffraction (reflection) methods or by using Powder X-ray diffraction (transmission) methods (PXRD). For crystalline solids having identical chemical composition, the different resulting crystal gratings are summarized by the term polymorphism. Regarding solvates, hydrates and polymorphs and salts with particular crystallinity reference is made to the international application WO2018/192973, which is included herein by reference.

In a further preferred aspect the present invention relates to the new use and method of treatment as defined herein, wherein the compounds of the formulae (I), (II) or (III) are selected from the group consisting of:

| Exp | |
|---|---|
| No. | Structure |
| 1 | 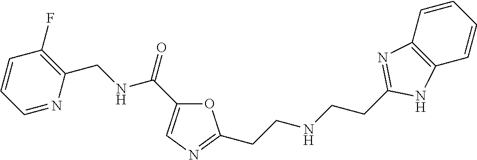 [SMILES: O=C(NCc1ncccc1F)c1cnc(CCNCCc2nc3ccccc3[nH]2)o1] |
| 2 | 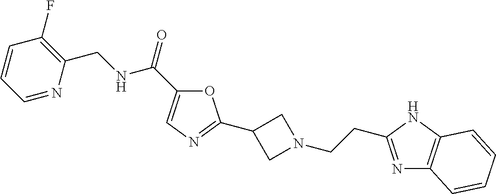 [SMILES: O=C(NCc1ncccc1F)c1cnc(C2CN(CCc3nc4ccccc4[nH]3)C2)o1] |
| 4 | 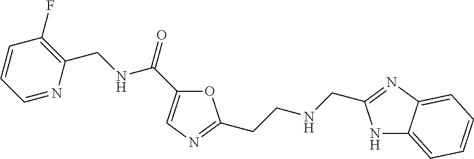 [SMILES: O=C(NCc1ncccc1F)c1cnc(CCNCc2nc3ccccc3[nH]2)o1] |
| 40 | 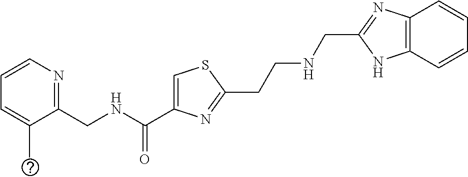 [SMILES: Cc1cccnc1CNC(=O)c1csc(CCNCc2nc3ccccc3[nH]2)n1] |
| 94 | 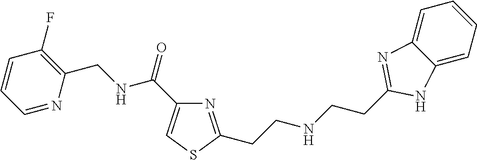 [SMILES: O=C(NCc1ncccc1F)c1csc(CCNCCc2nc3ccccc3[nH]2)n1] |
| 118 | 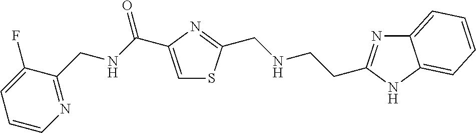 [SMILES: O=C(NCc1ncccc1F)c1csc(CNCCc2nc3ccccc3[nH]2)n1] |
| 126 | 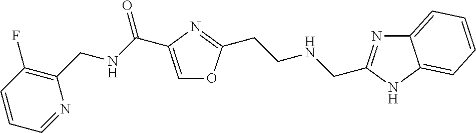 [SMILES: O=C(NCc1ncccc1F)c1coc(CCNCc2nc3ccccc3[nH]2)n1] |
| 127 | 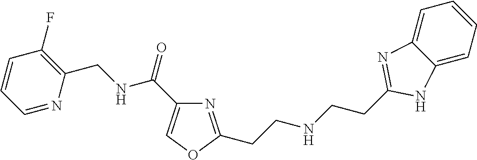 [SMILES: O=C(NCc1ncccc1F)c1coc(CCNCCc2nc3ccccc3[nH]2)n1] |
| 193 | 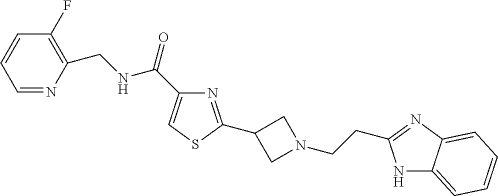 [SMILES: O=C(NCc1ncccc1F)c1csc(C2CN(CCc3nc4ccccc4[nH]3)C2)n1] |
| 206 | 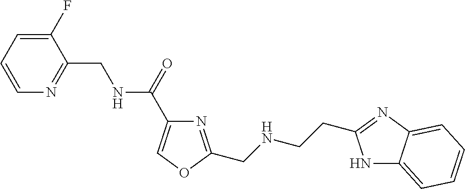 [SMILES: O=C(NCc1ncccc1F)c1coc(CNCCc2nc3ccccc3[nH]2)n1] |
| 208 | 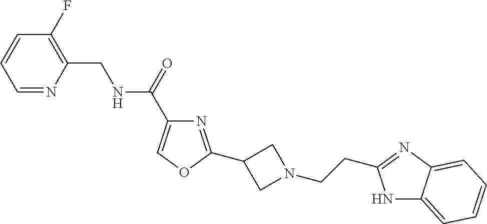 [SMILES: O=C(NCc1ncccc1F)c1coc(C2CN(CCc3nc4ccccc4[nH]3)C2)n1] |
| 233 | 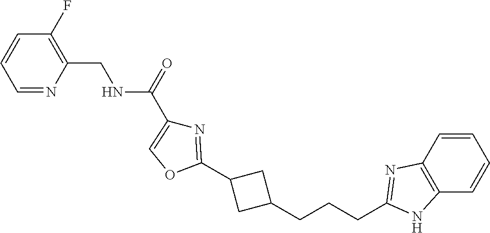 [SMILES: O=C(NCc1ncccc1F)c1coc(C2CC(CCCc3nc4ccccc4[nH]3)C2)n1] |

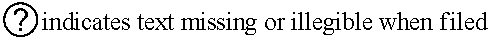

and its pharmaceutically acceptable salts, solvates, hydrates and polymorphs.

In a further preferred aspect the present invention relates to the new use and method of treatment as defined herein, wherein the compounds of the formulae (I), (II) or (III) are selected from the group consisting of:

| Exp. | |
|---|---|
| No. | Structure |
| 1 | 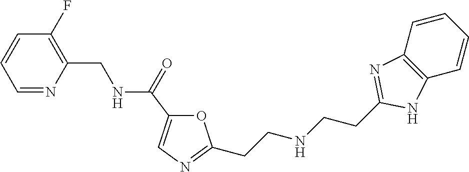 [SMILES: O=C(NCc1ncccc1F)c1cnc(CCNCCc2nc3ccccc3[nH]2)o1] |
| 40 | 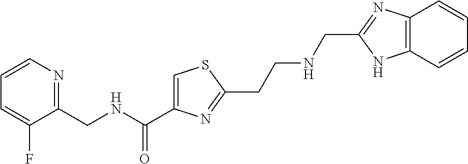 [SMILES: O=C(NCc1ncccc1F)c1csc(CCNCc2nc3ccccc3[nH]2)n1] |
| 94 | 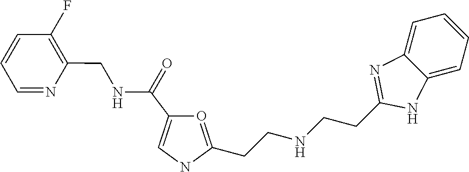 |
| 127 | 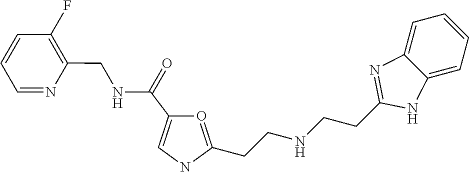 |
| 208 | 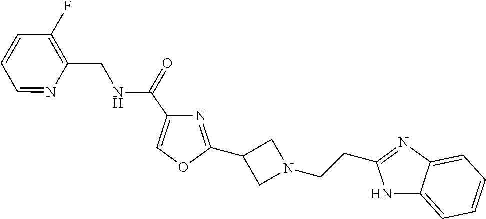 [SMILES: O=C(NCc1ncccc1F)c1coc(C2CN(CCc3nc4ccccc4[nH]3)C2)n1] |

and its pharmaceutically acceptable salts, solvates, hydrates and polymorphs.

In a further preferred aspect the present invention relates to the new use and method of treatment as defined herein, wherein the compounds of the formulae (I), (II) or (III) are selected from the group consisting of:

| Exp. | |
|---|---|
| No. | Structure |
| 1 | 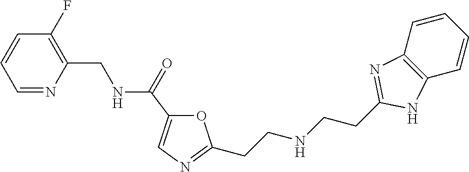 [SMILES: O=C(NCc1ncccc1F)c1cnc(CCNCCc2nc3ccccc3[nH]2)o1] |
| 2 | 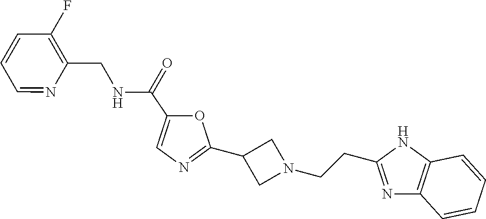 [SMILES: O=C(NCc1ncccc1F)c1cnc(C2CN(CCc3nc4ccccc4[nH]3)C2)o1] |
| 4 | 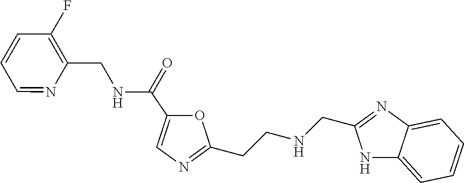 [SMILES: O=C(NCc1ncccc1F)c1cnc(CCNCc2nc3ccccc3[nH]2)o1] |
| 126 | 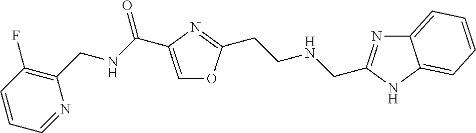 [SMILES: O=C(NCc1ncccc1F)c1coc(CCNCc2nc3ccccc3[nH]2)n1] |
| 127 | 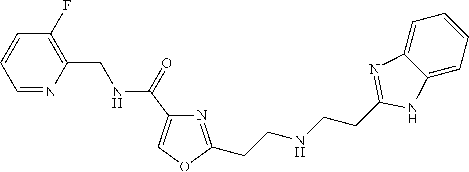 [SMILES: O=C(NCc1ncccc1F)c1coc(CCNCCc2nc3ccccc3[nH]2)n1] |
| 206 | 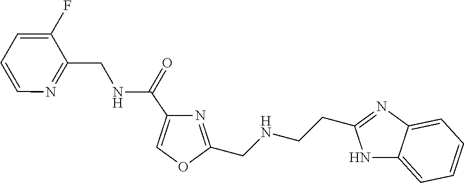 [SMILES: O=C(NCc1ncccc1F)c1coc(CNCCc2nc3ccccc3[nH]2)n1] |
| 208 | 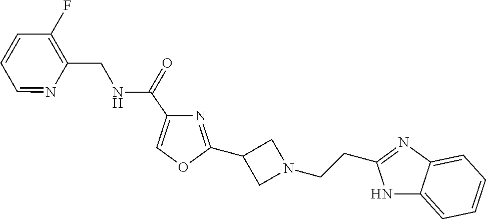 [SMILES: O=C(NCc1ncccc1F)c1coc(C2CN(CCc3nc4ccccc4[nH]3)C2)n1] |
| 233 | 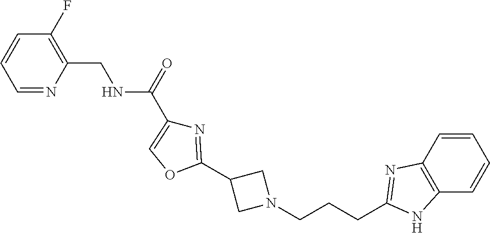 [SMILES: O=C(NCc1ncccc1F)c1coc(C2CN(CCCc3nc4ccccc4[nH]3)C2)n1] |

In a further preferred aspect the present invention relates to the new use and method of treatment as defined herein, wherein the compounds of the formulae (I), (II) or (III) are selected from the group consisting of:

| Exp. | |
|---|---|
| No. | Structure |
| 1 | 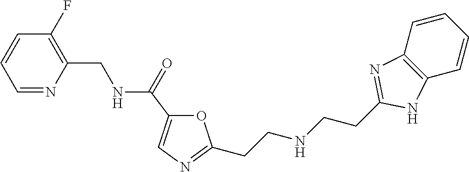 [SMILES: O=C(NCc1ncccc1F)c1cnc(CCNCCc2nc3ccccc3[nH]2)o1] |
| 127 | 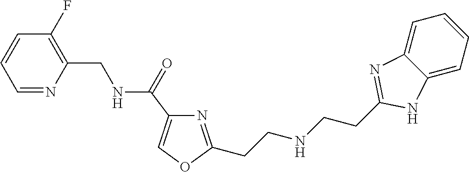 [SMILES: O=C(NCc1ncccc1F)c1coc(CCNCCc2nc3ccccc3[nH]2)n1] |
| 208 | 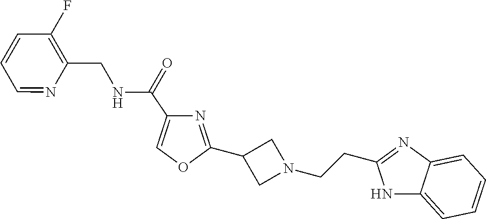 [SMILES: O=C(NCc1ncccc1F)c1coc(C2CN(CCc3nc4ccccc4[nH]3)C2)n1] |

and its pharmaceutically acceptable salts, solvates, hydrates and polymorphs.

In an even more preferred aspect of the invention the compounds of the formulae (I), (II) or (III) are selected from the group consisting of:

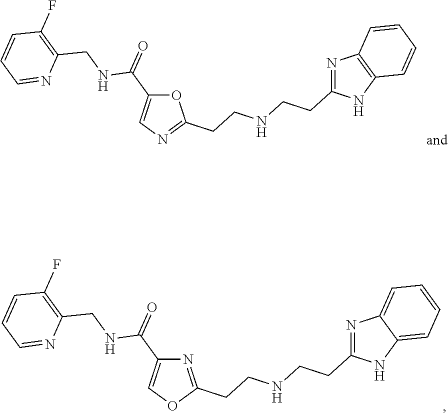 [SMILES: O=C(NCc1ncccc1F)c1cnc(CCNCCc2nc3ccccc3[nH]2)o1 ; O=C(NCc1ncccc1F)c1coc(CCNCCc2nc3ccccc3[nH]2)n1]

and its pharmaceutically acceptable salts, solvates, hydrates and polymorphs.

In a further preferred aspect of the invention the compounds of the formulae (I), (II) or (III) are selected from the group consisting of the following salts:

a 1:1 sulfate salt having the formula

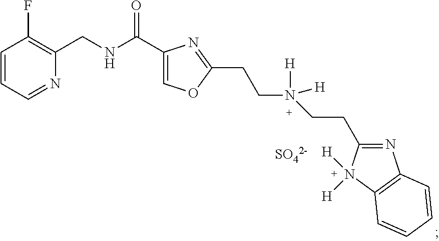

a 1:1 phosphate salt hay n the formula

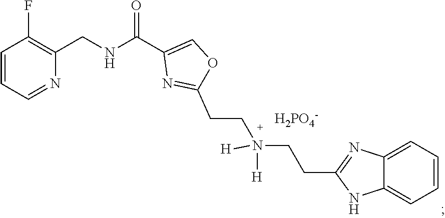

a 2:1 phosphate salt (hemiphosphate)

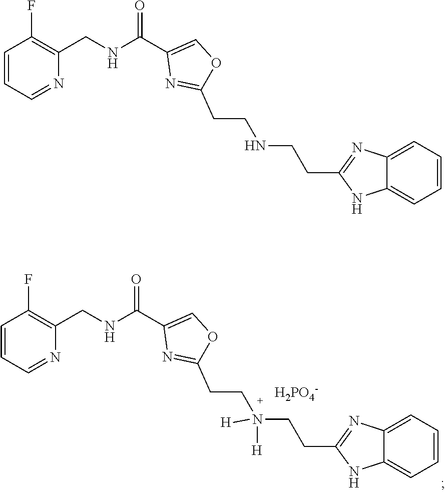

a 1:3 HCl salt having the formula

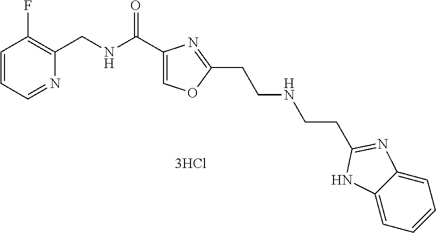 [SMILES: O=C(NCc1ncccc1F)c1coc(CCNCCc2nc3ccccc3[nH]2)n1]

and polymorphs thereof.

As described in WO2017/068089, WO2017/068090 and WO2018/192973 the compounds of the formula (I) act as ferroportin inhibitors. Regarding the ferroportin inhibitor activity of the compounds reference is thus made to said international applications.

### Medicaments Containing the Ferroportin Inhibitor Compounds

A further aspect of the invention relates to a medicament or a pharmaceutical composition containing one or more of the compounds of the formulae (I), (II) or (Ill) as defined anywhere herein for the new use and method of treatment of kidney injuries, such as in particular IRI and AKI, as defined anywhere herein.

Such medicament may further contain one or more pharmaceutical carriers and/or one or more auxiliaries and/or one or more solvents.

Preferably, the medicament is in the form of an oral dosage form, e.g. such as defined above.

Preferably the pharmaceutical carriers and/or auxiliaries and/or solvents are selected among suitable compounds for preparing oral and/or intravenous dosage forms.

The said pharmaceutical compositions contain, for example up to 99 weight-% or up to 90 weight-% or up to 80 weight-% or up to 70 weight-% of the ferroportin inhibitor compounds of the present invention, the remainder being each formed by pharmacologically acceptable carriers and/or auxiliaries and/or solvents and/or optionally further pharmaceutically active corn pounds.

Therein, the pharmaceutically acceptable carriers, auxiliary substances or solvents are common pharmaceutical carriers, auxiliary substances or solvents, including various organic or inorganic carrier and/or auxiliary materials as they are customarily used for pharmaceutical purposes, in particular for solid medicament formulations. Examples include excipients, such as saccharose, starch, mannitol, sorbitol, lactose, glucose, cellulose, talcum, calcium phosphate, calcium carbonate; binding agents, such as cellulose, methylcellulose, hydroxypropylcellulose, polypropyl pyrrolidone, gelatine, gum arabic, polyethylene glycol, saccharose, starch; disintegrating agents, such as starch, hydrolyzed starch, carboxymethylcellulose, calcium salt of carboxymethylcellulose, hydroxypropyl starch, sodium glycol starch, sodium bicarbonate, calcium phosphate, calcium citrate; lubricants, such as magnesium stearate, talcum, sodium laurylsulfate; flavorants, such as citric acid, menthol, glycin, orange powder; preserving agents, such as sodium benzoate, sodium bisulfite, paraben (for example methylparaben, ethylparaben, propylparaben, butylparaben); stabilizers, such as citric acid, sodium citrate, acetic acid and multicarboxylic acids from the titriplex series, such as, for example, diethylenetriaminepentaacetic acid (DTPA); suspending agents, such as methycellulose, polyvinyl pyrrolidone, aluminum stearate; dispersing agents; diluting agents, such as water, organic solvents; waxes, fats and oils, such as beeswax, cocoa butter; polyethylene glycol; white petrolatum; etc..

Liquid medicament formulations, such as solutions, suspensions and gels usually contain liquid carrier, such as water and/or pharmaceutically acceptable organic solvents. Furthermore, such liquid formulations can also contain pH-adjusting agents, emulsifiers or dispersing agents, buffering agents, preserving agents, wetting agents, gelatinizing agents (for example methylcellulose), dyes and/or flavouring agents, for example as defined above. The compositions may be isotonic, that is, they can have the same osmotic pressure as blood. The isotonicity of the composition can be adjusted by using sodium chloride and other pharmaceutically acceptable agents, such as, for example, dextrose, maltose, boric acid, sodium tartrate, propylene glycol and other inorganic or organic soluble substances. The viscosity of the liquid compositions can be adjusted by means of a pharmaceutically acceptable thickening agent, such as methylcellulose. Other suitable thickening agents include, for example, xanthan gum, carboxymethylcellulose, hydroxypropylcellulose, carbomer and the like. The preferred concentration of the thickening agent will depend on the agent selected.

Pharmaceutically acceptable preserving agents can be used in order to increase the storage life of the liquid composition. Benzyl alcohol can be suitable, even though a plurality of preserving agents including, for example, paraben, thimerosal, chlorobutanol and benzalkonium chloride can also be used.

### Combination Therapy

A further object of the present invention relates to medicaments or combined preparations containing one or more of the ferroportin inhibitor compounds as defined anywhere herein and at least one further pharmaceutically active compound (“combination therapy compound”), preferably an additional active compound being useful in the treatment of kidney injuries, such as in particular in IRI and AKI as defined herein. Preferred combination therapy compounds are in particular compounds used in the prophylaxis and treatment of iron overload and the associated symptoms. Most preferred combination therapy compounds are iron-chelating compounds, or compounds for the prophylaxis and treatment of any of the states, disorders or diseases accompanying or resulting from iron overload, IRI and AKI. Preferably, the at least one additional pharmaceutically active combination therapy compound is selected from drugs for reducing iron overload (e.g. Tmprss6-ASO) and iron chelators, in particular curcumin, SSP-004184, Deferitrin, deferasirox, deferoxamine and deferiprone.

Further preferred combination therapy compounds may be selected from drugs for treating inflammation, synthetic human hepcidin (LJPC-401), the hepcidin peptidomimetic PTG-300 and the anti-sense oligonucleotide targeting Tmprss6 (IONIS-TMPRSS6-L RX).

In a further aspect the present invention relates to the new use and medical treatment as defined herein, wherein the ferroportin inhibitor compounds as defined herein are administered to the patient in need thereof in a combination therapy with one or more of the combination therapy compounds defined above in a fixed dose or free dose combination for sequential use. Such a combination therapy comprises co-administration of the ferroportin inhibitor compounds as defined in the present invention with the at least one additional pharmaceutically active compound (drug/combination therapy compound).

Combination therapy in a fixed dose combination therapy comprises co-administration of the ferroportin inhibitor compounds as defined herein with the at least one additional pharmaceutically active compound in a fixed-dose formulation.

Combination therapy in a free dose combination therapy comprises co-administration of the ferroportin inhibitor compounds as defined herein and the at least one additional pharmaceutically active compound in free doses of the respective compounds, either by simultaneous administration of the individual compounds or by sequential use of the individual compounds distributed over a time period.

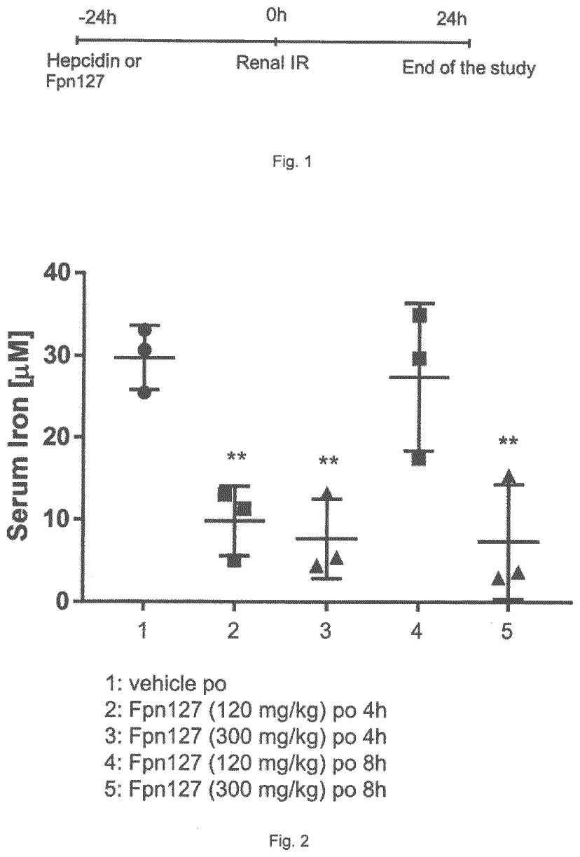

FIG. 1: Illustration of the dosing regimen in Example II

FIG. 2: Serum iron levels in naïve C57BL/6 mice treated with Fpn127 at 120 mg/kg or 300 mg/kg for 4 h or 8 h. Vehicle was 0.5% methylcellulose in water

### EXAMPLES

The invention is illustrated in more detail by the following examples. The examples are merely explanatory, and the person skilled in the art can extend the specific examples to further ferroportin inhibitor compounds according to the present invention.

### I. Ferroportin Inhibitor Example Compounds

Regarding the preparation of the specific Ferroportin Inhibitor Example Compounds Nos. 1, 2, 4, 40, 94, 118, 126, 127, 193, 206, 208 and 233 as described herein and the preparation of pharmaceutically acceptable salts thereof reference is made to the international applications WO2017/068089, WO2017/068090 and WO2018/192973.

Regarding the preparation of the specific Ferroportin Inhibitor Compounds described in WO2020/123850 A1 reference is made to the preparation methods described in said international application WO2020/123850 A1.

### II. Pharmacological Assays

### II. 1 Reduction of Serum Iron by Fpn127 in C57BL/6 Mice

To determine the dose of Fpn127 that causes sustained serum iron reduction, C57BL/6 mice received either 120 mg/kg or 300 mg/kg of Fpn127 po for 4 h or 8 h. Serum iron was reduced significantly by both doses at 4 h post dosing. However, only the dose of 300 mg/kg sustained the hypoferremia for 8 h (FIG. 2). These data in naïve C57BL/6 mice suggested to use the dose of 300 mg/kg po in the bilateral ureteral obstruction model of AKI.

### II. 2 In Vivo Efficacy of the Ferroportin Inhibitor Fpn127 in the Bilateral Ischemic Acute Kidney Injury Mouse Model

Renal ischemia-reperfusion injury (IRI) is a major cause of acute kidney injury (AKI) and iron-mediated oxidative stress by non-transferrin bound iron (NTBI) is implicated in IRI pathogenesis (Baliga R, Ueda N, Shah S V: Biochem J 291: 901-905, 1993). Hepcidin, the key regulator or iron homeostasis preventing iron export from cells via ferroportin, has been shown to mediate protection in renal IRI (Scindia Y et al, JASN, 2015).

The efficacy of the ferroportin inhibitor compounds of the present invention in treating kidney injuries, such as IRI and AKI, can be determined in a model of bilateral ischemic kidney injury. As an exemplary ferroportin inhibitor compound according to formula (I) Example Compound No. 127 (Fpn127) can be used.

To determine the optimal level of kidney injury in this model, a pilot study comparing 25 min and 30 min of bi-lateral renal ischemia is conducted, as described in Wei Q and Dong Z. “Mouse model of ischemic acute kidney injury: technical notes and tricks”, Am J Physiol Renal Physiol 303: F1487-F1494, 2012. The mouse is anesthetized with 50-60 mg/kg of pentobarbital sodium by intraperitoneal injection. Pentobarbital solution is diluted with sterile saline to have a concentration of 5 mg/ml for injection. Shortly after pentobarbital injection, 50 μg/kg of buprenorphine is administered subcutaneously for relief from pain and distress. After pentobarbital and buprenorphine injections, the hair on both sides of the mouse is removed with the hair clipper. The skin in the surgical area is then wiped clean with 70% alcohol swab. Immediately after the skin preparation, the mouse is placed on the homeothermic blanket of a homeothermic monitor system and covered by sterile gauze. The body temperature is monitored through a rectal probe and controlled in the range of 36.5-37° C. (our routine set-point is 36.7° C. and temperature varies in 0.1° C. range). Surgery will not be started until 1) the body temperature is stabilized at the set-point, and 2) the mouse is in deep anesthesia and thus does not respond to pain induced by toe pinch. It usually takes 30 min after pentobarbital injection to achieve deep anesthesia. The mouse is placed on the thermostatic station laying on the right side. The skin and muscle on the left flank side are cut open along the back to expose the left kidney. The incision is positioned at ⅓ of the body from the back of the mouse and the incision size is 1-1.5 cm along the back. The kidney is then pushed out from the cut with sterile cotton swabs to expose the renal pedicle. Dissection of the pedicle tissue is done with ultra-fine-point tweezers to remove the tissue around the renal pedicle to expose the blood vessels for renal pedicle clamping. After the preparation, the left kidney is returned to the abdomen cavity. The right renal pedicle is prepared by a similar surgical procedure, but the incision is closer to the rib due to the different position of the right kidney. After the pedicle preparation, both kidneys are returned back to their original positions in the abdomen cavity. The mouse is then covered with sterile gauze on the thermostatic station for its body temperature to stabilize again, which usually takes 5-10 min. The right kidney is gently pushed out of the body cavity with cotton swabs to expose the pedicle. A microaneurysm is used to clamp the pedicle to block the blood flow to the kidney to induce renal ischemia. The duration of right kidney ischemia starts from the time of clamping. Complete ischemia is indicated by color change of the kidney from red to dark purple in a few seconds. After verification of the kidney color changes, the kidney is returned to the abdomen cavity. The mouse is then laid on its right side for the left renal pedicle clamping and ischemia. There is around 1-1.5 min time latency between the right and left kidney clamping. However, the ischemic time of each side is recorded separately to ensure both kidneys receive the same durations of ischemia. After the ischemia, the micro-aneurysm clips are released at desired times for each kidney to start the reperfusion, which is indicated by the change of kidney color to red. A Vicryl suture is used to close the muscle layer of the incision followed by the closure of the skin wound with Michel wound clips. Immediately after the wound closure, 0.5 ml warm sterile saline is given intraperitoneally to each mouse. The animal is then kept on a heating pad until it gains full consciousness before being returned to its housing cage. Kidneys are exposed to reperfusion for 24 hours. Sham-operated mice undergo bilateral flank incisions without clamping of renal pedicles. 24 hours after ischemia, mice are euthanized and kidney and blood are collected. Serum creatinine levels are measured and serve as marker for severity of injury. The following groups of 8- to 10-week-old C578L/6J male mice; n=4/group are used in the pilot study:
1. Sham operated
2. IRI— 25 min
IRI— 30 min

### Main Study:

Based on the results from the pilot study, the ischemic duration with plasma creatinine levels between 2.5-3 mg/dL is selected for the main study. Mice are pre-treated with Fpn127 (300 mg/kg, per os, p.o), Fpn127 (100 mg/kg, intravenous, i.v.), Hepcidin (50 μg/mouse, intraperitoneal, i.p.), or vehicle (0.5% methylcellulose, p.o.) for 24 hours before IRI. Then, both renal pedicles are exposed and cross-clamped either for 25 min or 30 min in anaesthetized mice. Clamps are removed, and kidneys are allowed to reperfuse for 24 hours. Sham-operated mice undergo bilateral flank incisions without clamping of renal pedicles. 24 hours after ischemia, mice are euthanized and kidneys and blood are collected.

The following groups of mice are included: 8- to 10-week-old C57BL/6J male mice n=8/group.
4. Sham operated—vehicle (0.5% Methylcellulose, 10 ml/kg, p.o.)
5. IRI— vehicle, (0.5% Methylcellulose, 10 ml/kg, p.o.)
6. IRI— Fpn127 (300 mg/kg, 10 ml/kg, 24 h before IRI, p.o.)
7. IRI—Fpn127 (100 mg/kg, 5 ml/kg, 24 h before IRI, i.v.)
8. IRI— Hepcidin (50 μg/mouse, 5 ml/kg, 24 h before IRI), i.p.

The dosing time interval is illustrated in FIG. 1.

The following markers are measured in the end of the study: Plasma creatinine, blood urea nitrogen (BUN), total plasma iron, NTBI, plasma hepcidin, spleen, kidney and liver iron.

Hematoxylin/eosin (HE) staining of kidney sections is performed to evaluate the extent of kidney tissue injury with tubular injury score as readout. Immunohistochemistry using caspase-3 staining on kidney sections are performed to assess the level of kidney damage.

ROS-mediated oxidative stress in kidney is assessed by detection of 4-HNE in kidney sections. Ferroportin gene expression in liver, spleen and kidney is measured by qPCR.

H-Ferritin expression in organs is measured by western blot and qPCR.

Infiltration of leukocytes in kidney is detected by staining with anti-CD45 antibody using flow cytometry.

The neutrophils are identified by anti-Ly6G and Ly6C labeling of CD11b+ cells and flow cytometry analysis.

In patients, the onset of acute kidney injury is unforeseen and ideally drug dosing closer to the ischemia event would be preferable. To optimize the dosing regimen of the ferroportin inhibitor mice are treated with Fpn127 for 1, 3, 6, 9, 12 h and 15 h before the IRI. The following groups of mice are included: 8- to 10-week-old C57BL/6J male mice n=8/group.
1. Sham operated—vehicle (0.5% Methylcellulose, 10 ml/kg, p.o.)
2. IRI— vehicle, (0.5% Methylcellulose, 10 ml/kg, p.o.)
3. IRI— Fpn127 (300 mg/kg, 10 ml/kg, 2 h before IRI, p.o.)
4. IRI— Fpn127 (300 mg/kg, 10 ml/kg, 4 h before IRI, p.o.)
5. IRI Fpn127 (300 mg/kg, 10 ml/kg, 6 h before IRI, p.o.)
6. IRI Fpn127 (300 mg/kg, 10 ml/kg, 8 h before URI, p.o.)
7. IRI— Fpn127 (300 mg/kg, 10 ml/kg, 12 h before IRI, p.o.)
8. IRI— Fpn127 (300 mg/kg, 10 ml/kg, 16 h before IRI, p.o.)

The parameters of kidney function measured in the main study are used as efficacy readouts. To further optimize the dosing regimen mice are administered with Fpn127 via i.v. route for 0.5 h, 1 h and 3 h before IRI or 1 h after IRI. The following groups of mice are included: 8- to 10-week-old C57BL/6J male mice n=8/group.

Sham operated—vehicle (saline, 5 ml/kg, i.v.)
1. IRI vehicle, (saline, 5 ml/kg, i.v.))
2. IRI— Fpn127 (100 mg/kg, 5 ml/kg, 0.5 h before IRI, i.v.)
3. IRI— Fpn127 (100 mg/kg, 5 ml/kg, 1 h before IRI, i.v.)
4. IRI Fpn127 (100 mg/kg, 5 ml/kg, 3 h before IRI, i.v.)
5. IRI— Fpn127 (100 mg/kg, 5 ml/kg, 1 h after IRI, i.v.)

The parameters of kidney function measured in the main study are used as efficacy readouts.

### II. 3 Reduction of the Proportion of ROS in Kidney Tissue

The effect of the ferroportin inhibitor, e.g. of Fpn127, on ROS levels in kidney tissue can be monitored by commercially available far-red emitting ROS-sensitive sensor.

In particular, ROS determination can be used as an efficiency marker, similar as described in Scindia et al., 2015 (cited above).

### III. Effect of Fpn127 on NTBI and LPI Levels in the IRI/AKI Mouse Model Described Above

As described above, elevated plasma NTBI levels as a result of ferroportin-mediated export of iron from macrophages recycling damaged cells, such as RBCs and other types of damaged cells during AKI are considered to induce tissue injury. Dosing of ferroportin inhibitors of the present invention, such as Fpn127 has the potential to reduce the levels of plasma NTBI (and LPI) and the associated adverse effects.

The levels of NTBI in the mouse model of IRI/MAKI described above are investigated in mice treated with either vehicle or ferroportin inhibitors of the present invention, such as Fpn127, as indicated above. The nitrilotriacetate-NTBI method (NTA-NTBI) previously described (Singh S, Hider R C, Porter J B. “A direct method for quantification of non-transferrin-bound iron.“Anal Biochem. 1990 May 1; 186(2):320-3) is used with minor modifications.

Briefly, 0.02 mL of 800-mM NTA (at pH 5.7) is added to 0.18-mL mouse serum pool and allowed to stand for 30 minutes at 22° C. The solution is ultrafiltered using Whatman Vectaspin ultracentrifugation devices (30 kDa) at 12320 g and the ultrafiltrate (0.02 mL) injected directly onto an high-performance liquid chromatography column (ChromSpher-ODS, 5 μM, 100×3 mm, glass column fitted with an appropriate guard column) equilibrated with 5% acetonitrile and 3-mM deferiprone (DFP) in 5-mM MOPS (pH 7.8). The NTA-iron complex then exchanges to form the DFP-iron complex detected at 460 nm by a Waters 996 photodiode array. Injecting standard concentrations of iron prepared in 80-mM NTA is used to generate a standard curve. The 800-mM NTA solution used to treat the samples and prepare the standards is treated with 2-μM iron to normalize the background iron that contaminates reagents. This means that the zero standard gives a positive signal because it contains the added background iron as an NTA-complex. When unsaturated transferrin is present in sera, this additional background iron can be donated to vacant transferrin sites resulting in a loss of the background signal and yielding a negative NTBI value.

NTBI is also measured using an alternative method (CP851 bead-NTBI) assay as described in Garbowski M W, Ma Y, Fucharoen S, Srichairatanakool S, Hider R, Porter J B. “Clinical and methodological factors affecting non-transferrin-bound iron values using a novel fluorescent bead assay.” Transl Res. 2016). The standards for this assay are prepared as follows: 1-mM iron-NTA complex (1:2.5 molar ratio), prepared from 100-mM NTA and 18-mM atomic absorption standard iron solution, is diluted with MilliQ water to a final concentration between 0 and 100 μM. For the standard curve, 120 μL of probe-labeled bead suspensions are incubated with 20 μL of buffered NTA-iron solutions of known concentration for 20 minutes at room temperature, with subsequent addition of 20 μL control serum from wild type mice (without free iron) and 40-μL paraformaldehyde (10% in MOPS) at a final concentration of 2%. The suspensions in sealed 96-well plates are incubated at 37° C. for 16 hours with shaking before fluorescence measurement by flow-cytometry. For serum samples of unknown iron concentrations, 140 μL quantities of beads are incubated with 20 μL of serum samples for 20 minutes, with subsequent addition of 40-μL paraformaldehyde at a final concentration of 2%. The chelatable fluorescent beads are mixed with serum from wild type mice as a control to set up the fluorescence at 100% and the relative fluorescence of chelatable fluorescent beads with serum from mice tested in the IRI/AKI model described above was calculated accordingly. Measurements are performed on Beckman Coulter F C500 flow-cytometer and analysis on FlowJo software. Gates were based on dot-plots of untreated bead populations. Median fluorescence of 10,000 events was recorded and corrected for bead auto-fluorescence. A standard curve was fitted with variable-slope sigmoidal dose response function.

NTBI, which encompasses all forms of serum iron that are not tightly associated with transferrin, is chemically and functionally heterogeneous. LPI represents a component of NTBI that is both redox active and chelatable, capable of permeating into organs and inducing tissue iron overload. LPI assay (Esposito BP1, Breuer W, Sirankapracha P, Pootrakul P, Hershko C, Cabantchik Z I. “Labile plasma iron in iron overload: redox activity and susceptibility to chelation.” Blood. 2003) measures the iron-specific capacity of a given sample to produce ROS and is considered one of the most relevant reactive iron species involved in tissue injury, such as AKI.

FeROS™ LPI kit (Aferrix Ltd.) is used to measure LPI in sera of mice treated with either vehicle or ferroportin inhibitors of the present invention, such as Fpn127.

NTBI and LPI levels in mice tested in the IRI/AKI model have been found to serve as translational markers allowing to evaluate the efficiency of the ferroportin inhibitor therapy.

This model can also be used to optimally design the dosing regimen of ferroportin inhibitors (such as e.g. Fpn127) for treating IRI and AKI. Therewith an optimal combination therapy for AKI can be established using the ferroportin inhibitors of the present invention.

With the models and examples described above, it is possible to demonstrate the capacity of the ferroportin inhibitors of the present invention in preventing and improving IRI and AKI.

### IV. Serum Creatinine, Urine Albumin Excretion, BUN, NGAL, Hemoglobin (Hb), Kidney H-Ferritin, Total Plasma Iron, RBC Hemolysis, Renal Ferroportin and KIM-1

These parameters can be determined using conventional methods.

For example, iron levels in plasma can be determined by the MULTIGENT Iron assay (Abbott Diagnostics). Total organ iron and is determined by inductively coupled plasma-optical emission spectroscopy (ICP-OES) in rodent models or by magnetic resonance imaging in patients.

### V. Serum Hepcidin, IL-6, Nonheme Iron, Renal Neutrophil Infiltration

These parameters can be determined as described by Scindia et al., 2015 (cited above).

### VI. Tissue/Organ Iron Levels

Iron levels, such as, e.g., liver, spleen or kidney iron levels can be determined using conventional assay(s). For example, iron levels can be determined by magnetic resonance imaging.

### VII. Tissue Morphology and Histology/Tubular Necrosis and Apoptosis

Tissue morphology and histopathology, such as tubular necrosis and apoptosis, can be performed as described by Scindia et al., 2015 (cited above).

### VIII. Efficacy of the Ferroportin Inhibitor VIT-2653 (Example Compound No. 40) to Attenuate Renal Injury Following Red Blood Cell Transfusion in Guinea Pigs

The efficacy of the ferroportin inhibitor compounds of the present invention in the prevention and treatment of acute kidney injuries in accordance with the present invention has further been confirmed by the results of J. H. Baek et al. “Ferroportin inhibition attenuates plasma iron, oxidant stress, and renal injury following red blood cell transfusion in guinea pigs”; Transfusion 2020 March; 60(3):513-523.

Said experiments have been carried out by intravenously administering the small-molecule ferroportin inhibitor VIT-2653, corresponding to Example Compound No. 40 of the present invention and further confirm the findings of the present invention.

The NTBI and Hb levels following exchange transfusion were significantly improved by dosing of the ferroportin inhibitor.

Total iron in kidneys following transfusion can be reduced by dosing of the ferroportin inhibitor. The contribution of circulating Hb on renal iron loading and the subsequent effects on oxidative stress and cellular injury was evaluated revealing that dosing of the ferroportin inhibitor to transfused mice significantly reduced the occurrence of changes in plasma creatinine >0.3 mg/dL, which is used as indicator of early acute kidney injury (AKI).

The experimental details and study conditions and the concrete study results can be derived from the mentioned paper.

1-16. (canceled)

17. A method of prevention and/or treatment of kidney injuries comprising administering to a patient in need thereof, compounds according to formula (I): 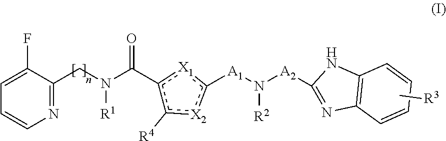
wherein
X¹ is N or O; and
X² is N, S or O;
with the proviso that X¹ and X² are different;
R¹ is selected from the group consisting of
hydrogen and
optionally substituted alkyl;
n is an integer of 1 to 3;
A¹ and A² are independently selected from the group of alkanediyl
R² is
hydrogen, or
optionally substituted alkyl;
or
A¹ and R² together with the nitrogen atom to which they are bonded form an optionally substituted 4- to 6-membered ring;
R³ indicates 1, 2 or 3 optional substituents, which may independently be selected from the group consisting of
halogen,
cyano,
optionally substituted alkyl,
optionally substituted alkoxy, and
a carboxyl group;
R⁴ is selected from the group consisting of
hydrogen,
halogen,
C₁-C₃-alkyl, and
halogen substituted alkyl;
and pharmaceutically acceptable salts, solvates, hydrates and polymorphs of any of the foregoing.

18. The method of claim 17, wherein the kidney injuries are selected from kidney injuries induced by catalytic free iron.

19. The method of claim 17, wherein the kidney injuries are selected from acute kidney injury (AKI), renal ischemia-reperfusion injury (IRI) and AKI caused by ischemic injury, AKI following surgery or surgical intervention, and kidney injury associated with red blood cell (RBC) transfusion.

20. The method of claim 17, wherein the kidney injuries are selected from renal ischemia-reperfusion injury (IRI), ischemic injury and acute kidney injuries.

21. The method of claim 17, wherein the patient in need thereof is suffering from at least one symptom selected from the group consisting of
(i) increased plasma creatinine levels,
(ii) increased urine albumin excretion,
(iii) decreased estimated glomerular filtration rate (eGFR),
wherein each, of (i), (ii) and (iii) are measured as compared to normal physiological levels, and
(iv) AKI.

22. The method of claim 17, wherein the patient in need thereof is at risk of suffering from AKI by any of the stages defined by the KDIGO or RIFLE/AKIN classification or by a CSA-NGAL score >0, or by an EGTI histology score >0.

23. The method of claim 17, wherein the prevention and/or treatment comprises at least one of
a) decrease, accelerated decrease or prevention of increase of serum creatinine,
b) increase or prevention of decrease of eGFR,
c) decrease or prevention of increase of renal ferroportin,
d) increase or prevention of decrease of H-ferritin levels,
e) decrease or prevention of increase of renal neutrophil infiltration, and
f) decrease or prevention of increase of serum IL-6 levels.

24. The method of claim 17, comprising administering to a patient in need thereof, wherein the patient in need thereof is at risk of at least one of IRI and AKI, compounds according to formula (I) one or more times within a time period of >0 to 48 hours, >0 to 36 hours, >0 to 24 hours, >0 to 20 hours, >0 to 18 hours, >0 to 16 hours, >0 to 12 hours, >0 to 10 hours, >0 to 8 hours, >0 to 6 hours, >0 to 5 hours, >0 to 4 hours, >0 to 3 hours, >0 to 2 hours, >0 to 1 hour, or >0 to 0.5 hours, prior to reperfusion, prior to red blood cell transfusion, prior to surgery or surgical intervention.

25. The method of claim 17, comprising administering to a patient in need thereof, compounds according to formula (I), one or more times within a time period between immediately after and up to 48 hours after a surgical intervention, RBC transfusion or an ischemic reperfusion event.

26. The method of claim 17, comprising administering to a patient in need thereof, compounds according to formula (I), one or more times within a time period between immediately after and up to 12 hours after a surgical intervention or an ischemic reperfusion event.

27. The method of claim 17, wherein the compounds of the formula (I) are administered in a dose between 0.5 to 500 mg, or between 1 to 300 mg, or between 1 to 250 mg, or between 0.001 to 35 mg/kg body weight.

28. The method of claim 17, wherein in formula (I)
n=1;
R³=hydrogen;
R⁴=hydrogen;
A¹=methylene or ethane-1,2-diyl;
A²=methylene, ethane-1,2-diyl or propane-1,3-diyl;
such that compounds according to formula (II) are defined: 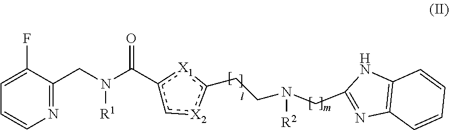
or
A¹ and R² together with the nitrogen atom to which they are bonded form an optionally substituted 4-membered ring, such that compounds according to formula (III) are defined: 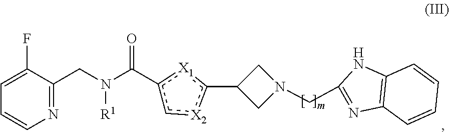
wherein in formula (II) and (III)
I is 0 or 1; and
m is an integer of 1, 2 or 3.

29. The method of claim 17, wherein the compounds of formula (I) are in the form of a pharmaceutically acceptable salt with at least one acid selected from the group consisting of benzoic acid, citric acid, fumaric acid, hydrochloric acid, lactic acid, malic acid, maleic acid, methanesulfonic acid, phosphoric acid, succinic acid, sulfuric acid, tartaric acid and toluenesulfonic acid, and solvates, hydrates and polymorphs of any of the foregoing.

30. The method of claim 28, wherein the compounds of formula (II) or (III) are in the form of a pharmaceutically acceptable salt with at least one acid selected from the group consisting of benzoic acid, citric acid, fumaric acid, hydrochloric acid, lactic acid, malic acid, maleic acid, methanesulfonic acid, phosphoric acid, succinic acid, sulfuric acid, tartaric acid and toluenesulfonic acid, and solvates, hydrates and polymorphs of any of the foregoing.

31. The method of claim 17, wherein the compounds of the formula (I) are selected from the group consisting of:

| Exp | |
|---|---|
| No. | Structure |
| 1 | 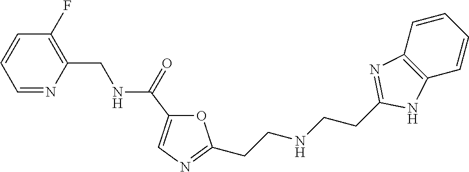 [SMILES: O=C(NCc1ncccc1F)c1cnc(CCNCCc2nc3ccccc3[nH]2)o1] |
| 2 | 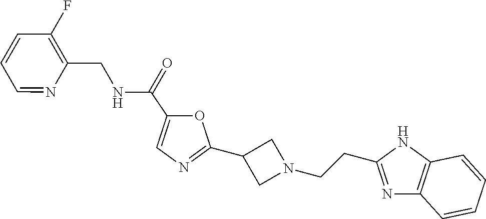 [SMILES: O=C(NCc1ncccc1F)c1cnc(C2CN(CCc3nc4ccccc4[nH]3)C2)o1] |
| 4 | 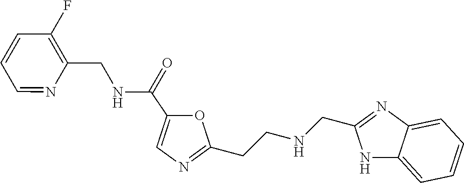 [SMILES: O=C(NCc1ncccc1F)c1cnc(CCNCc2nc3ccccc3[nH]2)o1] |
| 40 | 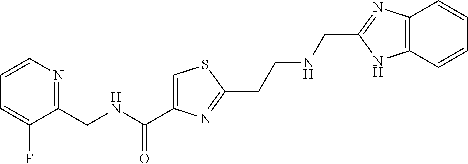 [SMILES: O=C(NCc1ncccc1F)c1csc(CCNCc2nc3ccccc3[nH]2)n1] |
| 94 | 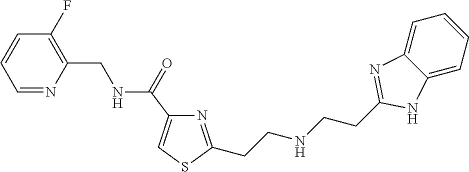 [SMILES: O=C(NCc1ncccc1F)c1csc(CCNCCc2nc3ccccc3[nH]2)n1] |
| 118 | 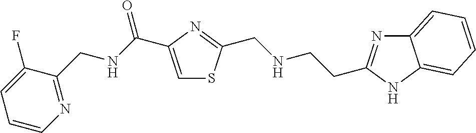 [SMILES: O=C(NCc1ncccc1F)c1csc(CNCCc2nc3ccccc3[nH]2)n1] |
| 126 | 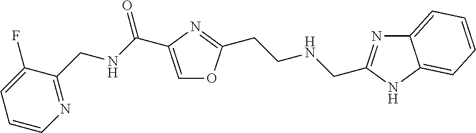 [SMILES: O=C(NCc1ncccc1F)c1coc(CCNCc2nc3ccccc3[nH]2)n1] |
| 127 | 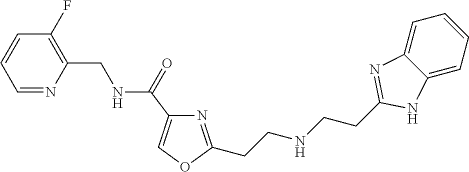 [SMILES: O=C(NCc1ncccc1F)c1coc(CCNCCc2nc3ccccc3[nH]2)n1] |
| 193 | 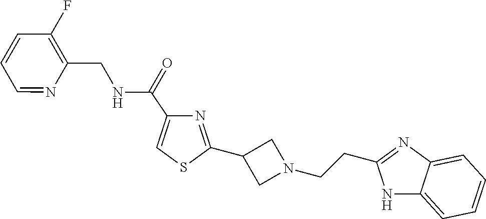 [SMILES: O=C(NCc1ncccc1F)c1csc(C2CN(CCc3nc4ccccc4[nH]3)C2)n1] |
| 206 | 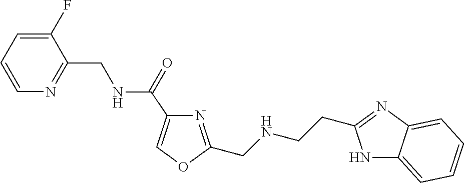 [SMILES: O=C(NCc1ncccc1F)c1coc(CNCCc2nc3ccccc3[nH]2)n1] |
| 208 | 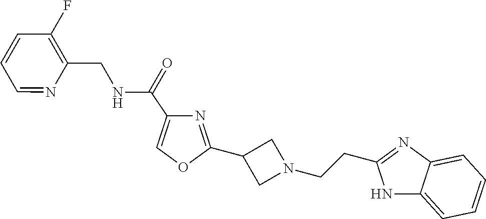 [SMILES: O=C(NCc1ncccc1F)c1coc(C2CN(CCc3nc4ccccc4[nH]3)C2)n1] |
| 233 | 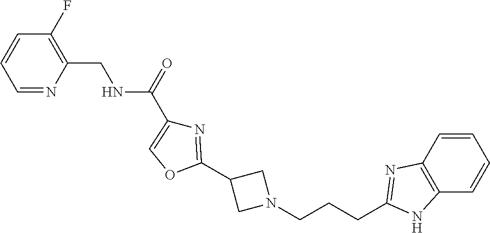 [SMILES: O=C(NCc1ncccc1F)c1coc(C2CN(CCCc3nc4ccccc4[nH]3)C2)n1] |

and pharmaceutically acceptable salts, solvates, hydrates and polymorphs of any of the foregoing.

32. The method of claim 17, wherein the compounds of the formula (I) are selected from the group consisting of:

| Exp. | |
|---|---|
| No. | Structure |
| 1 | 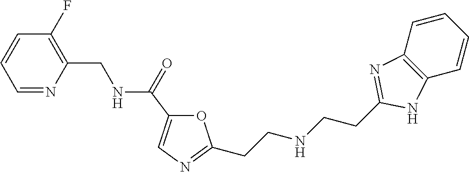 [SMILES: O=C(NCc1ncccc1F)c1cnc(CCNCCc2nc3ccccc3[nH]2)o1] |
| 40 | 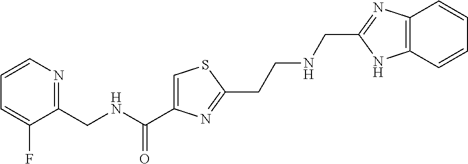 [SMILES: O=C(NCc1ncccc1F)c1csc(CCNCc2nc3ccccc3[nH]2)n1] |
| 94 | 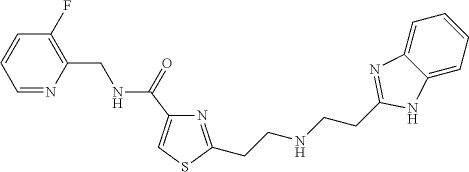 [SMILES: O=C(NCc1ncccc1F)c1csc(CCNCCc2nc3ccccc3[nH]2)n1] |
| 127 | 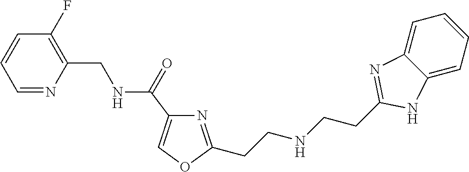 [SMILES: O=C(NCc1ncccc1F)c1coc(CCNCCc2nc3ccccc3[nH]2)n1] |
| 208 | 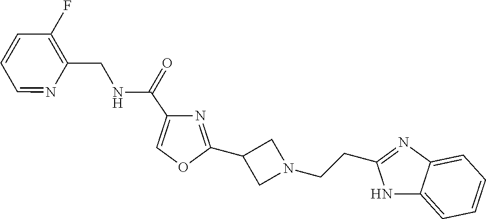 [SMILES: O=C(NCc1ncccc1F)c1coc(C2CN(CCc3nc4ccccc4[nH]3)C2)n1] |

and pharmaceutically acceptable salts, solvates, hydrates and polymorphs of any of the foregoing.

33. The method of claim 17, wherein the compounds of the formula (I) are selected from the group consisting of: 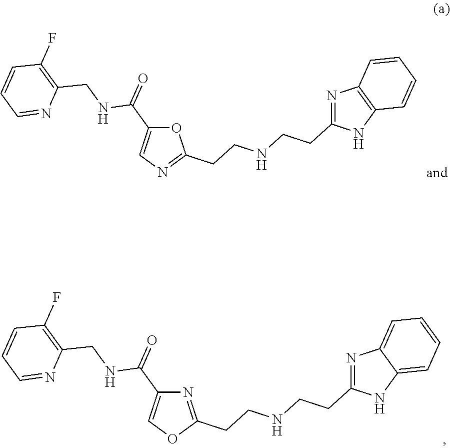 [SMILES: O=C(NCc1ncccc1F)c1cnc(CCNCCc2nc3ccccc3[nH]2)o1 ; O=C(NCc1ncccc1F)c1coc(CCNCCc2nc3ccccc3[nH]2)n1]
and pharmaceutically acceptable salts, solvates, hydrates and polymorphs of any of the foregoing;
(b) a 1:1 sulfate salt having the formula 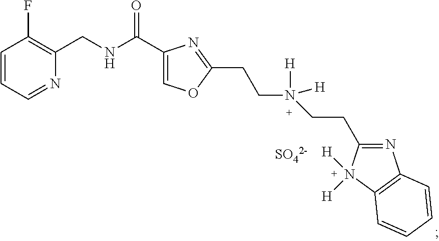
(c) a 1:1 phosphate salt having the formula 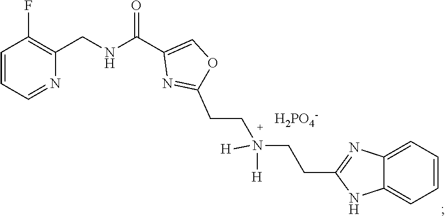
(d) a 1:3 HCl salt having the formula 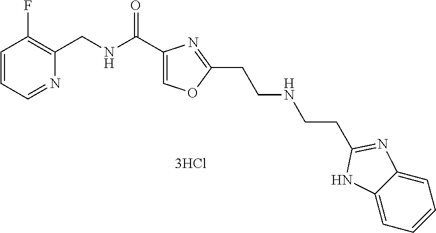 [SMILES: O=C(NCc1ncccc1F)c1coc(CCNCCc2nc3ccccc3[nH]2)n1]
and polymorphs of any of (b), (c) and (d).

34. The method of claim 17, wherein the compounds according to formula (I) are contained in a medicament, the medicament further comprising one or more pharmaceutical carriers and/or auxiliaries and/or solvents, and/or one or more additional pharmaceutically active compounds.

35. The method of claim 28, wherein the compounds according to formula (II) or (III) are contained in a medicament, the medicament further comprising one or more pharmaceutical carriers and/or auxiliaries and/or solvents, and/or one or more additional pharmaceutically active compounds.

36. The method of claim 17, wherein the method forms part of a combination therapy, wherein the combination therapy further comprises co-administration to the patient in need, one or more other pharmaceutically active compounds,
wherein the co-administration of the combination therapy is carried out in a fixed dose combination therapy by co-administration of the compounds according to formula (I) together with one or more other pharmaceutically active compounds in a fixed-dose formulation,
or
wherein the co-administration of the combination therapy is carried out in a free dose combination therapy by co-administration of the compounds according to formula (I) together with one or more other pharmaceutically active compounds in free doses of the respective compounds, either by simultaneous administration of the individual compounds or by sequential use of the individual compounds administered over a time period.
